(19) United States
(12) Patent Application Publication
(10) Pub. No.: US 2006/0182655 A1
(43) Pub. Date: Aug. 17, 2006

(54) Integrating analysis chip with minimized reactors and its application

(76) Inventors: Fanglin Zou, Chengdu City (CN); Chunsheng Chen, Chengdu City (CN); Ning Chen, Panzhihua City (CN); Jianxia Wang, Meishan City (CN)

Correspondence Address: BIRCH STEWART KOLASCH & BIRCH, PO BOX 747, FALLS CHURCH, VA, 22040-0747, US

(21) Appl. No.: 10/547,681

(22) PCT Filed: Mar. 4, 2004
(86) PCT No.: PCT/CN04/00169

§ 371 Date: Sep. 2, 2005

(30) Foreign Application Priority Data
Mar. 4, 2003 (CN) 03117397.7
Jun. 19, 2003 (CN) 03135251.0
Sep. 30, 2003 (CN) 03135958.2

(51) Int. Cl. G01N 33/00

(52) U.S. Cl. 422/58; 435/287.3

(57) ABSTRACT

The present invention involves a micro-array analysis-chip, especially multi-reactored micro-array analysis-chip. It also deals with the composition of the invented micro-array analysis-chip, especially the reactor structure with minimal height, including a partition structure, flow-path structure, and reaction-chamber structure, etc. Besides, it also involves a reactor-protecting system of the invented analysis-chip and marking system for the chip. Moreover, it also concerns with the application and related measuring device of the invented chip.

### FIELD OF THE INVENTION

The present invention relates to an analysis-chip, a kind with multiple reactors in particular. More specifically, to a highly integrated analysis-chip with minimal-height reactor. The present invention involves also application of the said analysis-chip.

### BACKGROUND ART

Analysis-chip, chip in short, has extensive application, including gene expression determination, gene screening, drug screening, diseases diagnosis and treatment, environmental monitoring and management, judicial identification etc.

The core of the chip is the reactor thereon, while the analysis-chip with markers also includes marking system. The three most important components of the reactor are: probe array, substrate and reactor structure. The probe array is formed by immobilizing probe-ligand used for analysis such as polypeptides, nucleic acids etc. onto the substrate surface through methods of spotting, printing etc. The substrate is usually a planar support containing active derivative groups, which is made of glass, metal, plastics or their derivatives in rectangle, round or other shapes. At present, the mainstream substrate in use is made chiefly of glass and its derivatives thereof (e.g. amino slides, aldehyde slides, epoxy slides and poly-amino acid-coated slides etc.) The reactor structure includes partition structure, flow-path structure, separating structure, chamber structure, etc.

The rapid development of analysis-chip is closely related to its high integration technology, which involves the following four facets: device miniaturization, high-density probe spots, multi-function integration, and high-density reactor. In the development of the high-integrated chip, significant progress has been made in the first three facets (e.g. analysis-chip with high-density of probe spots, chip-on-lab, etc). However, some problems rest. For example, in the polypeptide analysis-chip, only a few of different types of probe-ligand are needed, so what is the significance of the “high density”? Another problem is: in the extensive diagnosis and screening, test device at low cost is needed, and then how could the “multi-function” be achieved at a low cost? The inventors of the present invention hold that chips with high-integration at low cost remain one of the important facets of analysis-chip development.

The Current Situation of Analysis-Chip Development:

1. Analysis-Chip and Reactor Partition Structure

The foremost-developed chip, which is still in wide use today, is an analysis-chip with no partition structure. For example, an open non-flow analysis-chip with a single reactor is made through activation of a microscope glass slide and spotting the probe-ligands thereon, without other additional structures. It is not necessary to fix many types of probes on every reactor, when only a few types (e.g., less than 100 types) of targets are to be detected in a sample. Therefore, it is ratio between the area of the substrate probe region and the average area of the reactor substrate. In order to reduce the analysis cost, it is required to develop an analysis-chip with a high density of reactors. However, one of the main problems to be solved for the development is the selection of appropriate reactor partition structures. In addition, in order to reduce volume of subjected sample, it is necessary to limit the flow of liquid media. Moreover, the thickness of reactor partition structure has to be restricted on account of wide application of the optical scanning equipment. So, the partition structure with a height closing to zero will make smaller technical requirements on the scanning equipment. Meanwhile, the minimization of partition structure in height can also facilitate reactor washing.

Some chips with partition structure have been developed, which can be divided into two types: the multi-reactor chip with detachable partition-structure and the multi-reactor chip with undetectable partition-structure. Now an available type with undetectable partition structure is an analysis-chip with multi-open non-flow reactors. Its basic structure is: upon a standard substrate with a dimension of 25 mm×75 mm or 26 mm×76 mm (width×height), hydrophobic materials (water contact angle is 50-75°, e.g. polyester or PVC) are used to form a convex with a height more than 0.5 mm, and a width more than 1 mm. Thereby several open reactors in round or square shape are formed. So, in this chip, the flow of liquid media is still under control on the basis of the height difference between the partition structure and the plane of the substrate. In this case, if the partition area among open reactors is too low, cross-contamination with neighbor reactors will occur; however if it is too high, it will make a higher technical requirement on the biochip scanner. Besides, another type of analysis-chip available at present is based on the structure of ELISA plate with a detachable partition structure, whose main drawbacks lie in its complicated operation or the difficulty in minimization of its height or thickness of partition structures. So, how to develop an easily manufactured partition structure with minimal height at low cost remains one of the concerns among analysis-chip developers.

2. Analysis-Chip and Reactor Flow-Path Structure and Reactor Separation Structure

At present, micro-channel path, or micro-channel is the chief flow-path structure and separation structure on analysis-chip. The micro-channel, actually a concave flow-path, is usually less than 0.10 mm in width and less than 0.025 mm in depth. The analysis-chip with micro-channel path is called micro-channelled chip, e.g. the analysis-chip of Caliper Technologies Inc. (www.caliper.com). The advantages of micro-channelled analysis-chip are its high sensitivity and high speed, whereas the disadvantages are as follows:

1) because the micro-channel has to be etched beforehand, then after probe is deposited thereon, the micro-channel has to be sealed. With such a complicated structure, the process of industrialization is very difficult;

2) in detection, the liquid flow speed has to be controlled by some sophisticated instruments, e.g. electronic osmosis device;

3) after reaction, because probe molecules are fixed on the inner surface, e.g. fluorescence-labeled detection, the test results cannot be read directly with a common biochip scanner. Despite various projects on micro-channel path recently (e.g. U.S. Pat. Nos. 6,176,962 and 6,180,536), how to develop an easily manufactured flow-path structure at low cost remains one of the concerns.

3. Analysis-Chip and Reaction Structure

The reaction structure of analysis-chip includes open reaction cell and closed reaction-chamber. The analysis-chip with reaction-chamber is a closed chip, though parts of partition structure are still open. The closed analysis-chip includes flow and non-flow ones. In the present analysis-chip, ligand is immobilized only on one of two planes of the reaction-chamber, that does not favorite the sensitivity augmentation. Especially, for the existing flow analysis-chip with the closed chamber, the dynamic conditions for ligand-target reaction and conditions for observing signal are the factors taken into concern while designing. In present analysis-chip, the movement of liquid media in the reactor, especially in the reaction-chamber, is achieved through: mechanic transport, weight of liquid media itself, hydrophilicity of inlet structure or/and outlet structure, or their combinations. For the reaction-chamber, all of these are external liquid-delivering impetus. Of course, the top plane, bottom plane and wall of reaction-chamber usually possess hydrophilicity; but this kind of external liquid-delivering impetus of hydrophilicity only is weak. Consequently, on the recent closed microarray chip, while going through a test, especially through the operations of media change, sometimes the distribution of liquid media in the reaction-chamber of reactor is insufficient. For example, when gas exists, it will inhibit the distribution of liquid media, thereby interfering test results.

4. Analysis-Chip and Marking System

As for the current chips, one type requires pre-deposited markers in the reactor, in which the marker is released instantly in testing, whereas the other type requires that the marker solution be subjected to samples or to the reactor after probe-target reaction is completed. The latter, though of higher sensitivity, is difficult to operate. The former is easier to execute, but its test sensitivity is greatly decreased due to the Hook effect etc. When the former is to be adopted in analysis-chip analysis, in order to avoid Hook effect, all the markers should be in liquid status, and subjected into reaction device completely through a mechanic system if necessary. For example, when the planar analysis-chip with an outlaid marking system and a channeled analysis-chip with an inlayed marking system are employed in analysis, the special marker solution subjecting procedure and the corresponding mechanic system are required. In other words, in the prior analysis-chip analysis, since the labeling procedure is to be carried out with a mechanic system which helps deliver markers, the special marker container, marker-delivering channel, converter for the delivered material and converting procedure are usually indispensable, thus making the test device complicated. Besides, since only a small amount of marker is needed in detection, but its storage and preparation in both marking systems are very troublesome. Though the prior micro-channeled analysis-chip is equipped with an inlayed marking system, markers are released instantly in detection, therefore special procedure for adding the marker solution and corresponding mechanic system are required.

5. Reactor-Protecting System

As chip with high-density of reactors is introduced, one important but often neglected problem in the applications is: if only M out of N reactors (N>M) are used in testing, how to preserve the extra reactors for later use? The solution to this problem is the prerequisite for developing chips with high-density of reactors at low-cost. As for the analysis-chip with a single reactor, the reactor-protecting system at present is actually a protecting system for the whole chip, which usually is a plastic box containing analysis-chip racks. But strangely, the analysis-chip with multi-reactors now in use is equipped with no special protecting structure but said protecting plastic box for the analysis-chip with a single reactor.

Thus, the analysis-chip constituted by the present reactor partition structure, reactor flow-path structure, reactor separating structure, marking system and reactor-protecting system are yet to be improved.

### DISCLOSURE OF THE INVENTION

### SUMMARY OF THE INVENTION

The primary object of the present invention is to provide a highly integrated analysis-chip and the corresponding devices, which should be of easy preparation, lower cost, high sensitivity, small sample consumption, simple operation, and homogeneous distribution of reaction media. In fact, the improvement of integration of the analysis-chip is one of the keys to the solution. In addition, it is also very important to simplify operation procedures, facilitate washing, lower the height of the reactor structure and consequently reduce the investment on scanners by lowering the technical requirements. Hence, “minimizing the height of the reactor structure and maximizing the integration level” become our important research subjects, which results in the present invention.

So, the first embodiment of the present invention provides an analysis-chip, which comprises one or more reactors with minimal height, wherein said reactor comprises at least:

1). one or more striped-capillary reaction-chambers, wherein said reaction-chamber comprises:

i). top plane and bottom plane with a width more than 600 μm;

ii). probe-ligand and substrate probe-region in which said probe-ligand is immobilized;

iii). closed partition-structure with a height of 1-1000 μm, optimally 1-500 μm; and

iv). inlet and outlet,

2). reactor structure with minimal-height; which comprises at least open partition-structure comprising:

i). substrate blank region with a width of 0.5-10 mm; or/and

ii) one or more following convexes with a height less than 1000 μm, optimally less than 500 μm: hydrophobic convex, highly-hydrophobic convex, and water-absorbing convex; and optionally,

iii). convex flow-path comprising high-hydrophilic convex with a width of 5-4000 μm, and a height of 0.05-1000 μm, optimally 0.05-500 μm relative to said substrate probe region; and optionally, reactor-protecting structure comprising protective unit with a distance less than 1000 μm, optimally less than 500 μm from said substrate probe-region; and optionally,

3). marking-system convex, a marker-containing convex that does not cover said probe-ligand, wherein:

(1). said top plane and bottom plane are parts of top unit and bottom unit of said striped-capillary reaction-chamber, respectively;

(2). said substrate probe-region is on said top plane or/and bottom plane;

(3). said closed partition-structure is placed between said top plane and bottom plane;

(4). material and dimension of said planes, and distance between said top plane and bottom plane are such that the capillary phenomenon of analysis media can take place in said striped-capillary reaction-chamber;

(5). said substrate blank region and substrate probe region are on the same plane of a same substrate, which presents a water-absorptivity less than 0.1 g/g and a water contact angle as 40-80°;

(6). said highly-hydrophobic convex contains, at least in its partial surface, highly-hydrophobic material, wherein said highly-hydrophobic material presents a water contact angle of 40° bigger than that of said substrate probe region;

(7). said hydrophobic convex contains, at least in its partial surface, hydrophobic material, wherein said hydrophobic material presents a water contact angle as 55-80°;

(8). said water-absorbing convex contains, at least in its partial surface, water-absorbing material, wherein said water-absorbing material presents a water-absorptivity more than 0.1 g/g;

(9). said highly-hydrophilic convex contains, at least in its partial surface, highly-hydrophilic material, wherein said highly-hydrophilic material presents a water contact angle less than 40°; and

(10). said protective unit closes at least partially said reactor structure when no sample is subjected, and it is irreversibly removed completely or partially when sample is to be subjected.

The striped-capillary reaction-chamber of analysis-chip of the first or second embodiments of the present invention differs from the reaction-chamber of the prior closed-flow-chip: the former is characterized by its requirement on the selection of material and dimension of said planes, and distance between said top plane and bottom plane, so that the capillary phenomenon of analysis media can take place in said striped-capillary reaction-chamber. Whereas the latter lacks such requirements which make it necessary to introduce other media distribution mechanism, such as putting a membrane in the chamber etc.

The striped-capillary reaction-chamber of the present invention differs from the reaction-chamber of the prior micro-channeled analysis-chip: 1). the former presents wider substrate probe region (>500 μm) for immobilizing bigger probe array, whereas the latter narrow substrate probe plane (<500 μm); 2) the former can apply the capillarity with much bigger flow for direct media distribution; but the latter applies the capillarity with only tiny flow, which requires additional energy for media distribution.

The striped-capillary reaction-chamber of the present invention differs from the prior capillary analysis-chip: the capillary structure of the former is formed by two planes as well as the structure between the two planes, which make it possible to fix a bigger probe array there, whereas the latter couldn't.

The top plane and bottom plane of the striped-capillary reaction-chamber of the analysis-chip of the present invention could be in various geometric figures like rectangular, round etc. The materials of the top plane or/and bottom plane, especially the material for the substrate probe region could be selected from one of the following or their optional combinations: glass, silicon and silicide, metal oxide, metals and polymer materials as well as their derivatives etc. The probe array can be in various ordered distributions like matrix, broken lines, real lines etc. In fact, according to the various applications of chips, it can have and must have various selections of ordered ligand-distribution patterns. Said probes can be selected from one material of the following groups or their optional combinations: antigens, antibodies, ligand, aptamer screened by Systematic Evolution of Ligands by Exponential Enrichment (SELEX), polypeptides, single-strand or multi-strand DNA, nucleotides, polynucleotides, saccharides, coenzymes, cofactors, antibiotics, steroids, viruses and cells.

In one analysis-chip of the first embodiment of the present invention, said convex is formed by solidifying liquid substance or/and fixing solid substance onto surface of said chip, wherein: 1). said liquid substance includes solution, paint, gel, emulsion which contain said highly-hydrophobic, hydrophobic, highly-hydrophilic and water-absorbing materials respectively; and 2). said solid substance includes plate, film, board, tape, and powder which contain said highly-hydrophobic, hydrophobic, highly-hydrophilic and water-absorbing materials respectively.

In fact, in the present invention, said highly-hydrophobic convex, hydrophobic convex, highly-hydrophilic convex, water-absorbing convex are made respectively through solidifying liquid material onto the analysis-chip surface, or/and immobilizing solid substance onto the analysis-chip surface. Said liquid materials include solution, paint, gel or emulsion containing highly-hydrophobic material, hydrophobic material, highly-hydrophilic material, and water-absorbing material respectively. Said solid substances include plates, films, boards, tapes or powder containing highly-hydrophobic material, hydrophobic material, highly-hydrophilic material, and water-absorbing material respectively. The following example indicates how said convex is prepared through solidifying liquid material: A). provide the liquid material that has affinity to the substrate; B). coat it onto the specified position of the substrate; C). solidify the coating through drying or/and adding solidifying agents, wherein the solidifying reactions include oxidation polymerization (e.g. in case of the modified paint with drying oil etc.), solvent volatilization (e.g. in case of varnish dried and solidified at normal temperature etc.), solidification through using solidifying agent, solidification through heating, catalysis through bio-enzyme (e.g. nature lacquer etc.) and so on. The following example indicates how said convex is prepared through immobilizing solid material: A). provide the shaped solid substance which can bind to the substrate; B). bind the solid substance onto the specified position of the substrate through adhesion or/and thermal fusion.

The second embodiment of the present invention provides an analysis-chip, which comprises one or more flow or non-flow reactors with minimal-height, wherein said reactor comprises at least one or more striped-capillary reaction-chambers with a height of less than 100 μm, optimally 500 μm, and optionally, reactor-protecting structure, wherein said reaction-chamber comprises:

1). top plane and bottom plane with a width more than 600 μm;

2). probe-ligand and substrate probe-region in which said probe-ligand is immobilized;

3). closed partition-structure with a height of 1-1000 μm, optimally 1-500 μm; and 4). inlet and outlet, wherin:

(1). said top plane and bottom plane are parts of top unit and bottom unit of said striped-capillary reaction-chamber, respectively;

(2). said substrate probe-region is on said top plane or/and bottom plane;

(3). said closed partition-structure is placed between said top plane and bottom plane;

(4). material and dimension of said planes, and distance between said top plane and bottom plane are such that the capillary phenomenon of analysis media can take place in said striped-capillary reaction-chamber; and

(5). said substrate probe-region presents a water-absorptivity less than 0.1 g/g and a water contact angle as 40-80°.

In fact, the chip of the second embodiment is the analysis-chip with striped capillary reaction-chamber. Said reactor is flow or non-flow one. The situation of probe immobilization includes three patterns: the probe is immobilized on the top-plane substrate, immobilized on the bottom plane substrate, or immobilized respectively on the top-plane and bottom plane substrates, wherein the top plane is on the top unit, and the bottom plane, bottom unit. Positions of the inlet and the outlet include at least three patterns: they are on bottom unit, on top unit, and in between the bottom and top units. For the differences between the analysis-chip with striped capillary reaction-chamber of the second embodiment and the prior closed flow chip, micro-channeled chip, and capillary chip, please refer to that in the first embodiment for details.

In one analysis-chip of the first embodiment or the second embodiment of the present invention:

1). said closed partition-structure presents a height of 1-300 μm, optimally 30-100 μm;

2). said top plane presents a width of 1000-15000 μm and a length more than 1000 μm; and

3). said bottom plane presents a width of 1000-15000 μm and a length more than 1000 μm.

In the striped-capillary reaction-chamber, the width of said inlet or outlet may be equal to or not equal to the width of the top plane or bottom plane, and their heights may be equal to or not equal to the space between said top plane and bottom plane. We have discovered, in the situation of utilizing capillarity to distribute media directly, it is necessary to maintain certain chamber height (e.g. 30 μm). Besides, we have also discovered, when using mechanic force to distribute reaction media, it is beneficial to maintain certain height of chamber for avoiding uneven distribution, such as air bubble, etc.

In one analysis-chip of the first embodiment or the second embodiment of the present invention, the striped-capillary reaction-chamber presents a width of the top plane and bottom plane are 1500-15000 μm, preferably 2500-15000 μm.

In one analysis-chip of the first embodiment or the second embodiment of the present invention, said closed partition structure includes one or more of the following reversible or irreversible enclosing structures:

1). thermal enclosing structure;

2). chemical enclosing structure;

3). reversible or irreversible adhesive layer;

4). highly-hydrophobic layer; and

5). mechanic enclosing unit including coating, plate or tape of elastic polymer.

Said closure mainly refers to that of borders between top and bottom units except inlet and outlet. The closure is mainly the irreversible closure formed by the ways including adhesion or/and heat-fusion, or/and HF or sodium silicate linkage at low temperatures. The closure of adhesion can be irreversible closure or reversible closure. The closure formed by highly-hydrophobic water-partition seal and mechanic seal mainly is reversible closure. For irreversible closure, if necessary, the top or bottom planes can be opened by mechanical force. Said mechanical seal units all possess a certain elasticity, for example, their Shore hardness are between 20-100°, and are made from rubber (e.g. natural rubber and the derivatives thereof; synthetic rubbers like acrylonitrile-butadiene rubber, butyl rubber, fluororubber, silicone rubber, fluorosilicone rubber etc.) and various organic polymers (e.g. organosilicon polymer, fluoropolymer, polycarbonate, plastic etc.).

In one analysis-chip of the first embodiment or the second embodiment of the present invention, said top unit or/and bottom unit of said reaction-chamber present a thickness less than 1 mm, optimally less than 0.2 mm, and a detecting-light transparency rate more than 90%. One example for the transparent unit is a cover glass or activated cover glass, whose height is 0.15 mm. Hence the test result on the invented analysis-chip can be visualized directly with a signal-testing instrument. Said signal-testing instrument includes laser confocal scanners and laser scanners. In the embodiments, when the cover glass of the top unit is thin enough (e.g. ≦0.15 mm), it is not necessary to dismantle the invented analysis-chip before the scanning, for there is no obvious difference in the results with a glass cover or not.

In one analysis-chip of the first embodiment or the second embodiment of the present invention, said probe-ligand is immobilized on either said top plane or said bottom plane, wherein said plane with said immobilized probe-ligand presents hydrophilic property whereas said plane without said immobilized probe-ligand presents hydrophobic property.

In one analysis-chip of the first embodiment or the second embodiment of the present invention, said top plane is made of glass material whereas said bottom plane is made of hydrophilic or hydrophobic plastic.

In one analysis-chip of the first embodiment or the second embodiment of the present invention, one or more said probe-ligands are immobilized in one area whereas one or more ligate of said ligands are immobilized in one other area in said probe region, wherein said ligand and ligate include antigen and antibody. So this invented chip provides a new tool with high sensitivity, which can be used for detecting antigens as well as the corresponding antibodies simultaneously,

The third embodiment of the present invention provides an analysis-chip, which comprises one or more open reactors with minimal-height, wherein said reactor comprises probe-ligand, substrate probe region, open partition-structure with minimal height, and optionally, reactor-protectilg structure, wherein said open partition-structure comprises:

1). substrate blank region with a width of 0.5-10 mm;

2). highly-hydrophobic convex or/and water-absorbing convex, all of which presents a height less than 1000 μm, optimally less than 500 μm relative to said substrate probe region, wherein:

(1). said substrate blank region and substrate probe region are on the same plane of a same substrate, which present a water contact angle as 40-80°;

(2). said highly-hydrophobic convex contains, at least in its partial surface, highly-hydrophobic material, wherein said highly-hydrophobic material presents a water contact angle of 40° bigger than that of said substrate probe region; and

(3). said water-absorbing convex contains, at least in its partial surface, water-absorbing material, wherein said water-absorbing material presents a water-absorptivity more than 0.1 g/g.

In fact, the analysis-chip of the third embodiment includes the following chips of the present invention: the analysis-chip with highly-hydrophobic partition structure, the analysis-chip with water-absorbing partition structure and the analysis-chip with highly-hydrophobic/water-absorbing partition structure. The example for highly-hydrophobic/water-absorbing complex convex is: the highly-hydrophobic convex is near the substrate probe region whereas the water-absorbing convex is away from it. The convex herein can form various geometrical figures on the surface of substrate, such as single lines, multi-lines, strips, lattices etc. The invented open partition-structure with minimal height can be applied to all types of chips, such as the analysis-chip whose smallest space among reacting wells is 20% longer or more than the width of the reacting wells on its outstretched line; such as the analysis-chip with feeding structure or/and liquid-out structure; such as the analysis-chip with one or more structures that control the directional flow speed of the reaction media on the inlet and outlet area of the reactor; such as double-plane analysis-chip etc.

The invented open partition-structure with minimal height contains the substrate blank region with a width bigger than 0.2 mm, located on the same plane of the substrate probe region, which is very important and indispensable for chips. Differing from the devices like the microwell plate for ELISA immobilized with one type of probe, the reactors of the invented analysis-chip are usually immobilized with the arrays of varied types of probes therein, demanding the uniformity of the reaction circumstances for every probe in the array. But the existence of highly-hydrophobic convex or water-absorbing convex or highly-hydrophobic/water-absorbing complex convex will change the reaction condition in its peripheral. Therefore, it is necessary to make a region between the substrate probe region and the convex, in which no efficacious probe is immobilized (the substrate blank region). Through numerous experiments, we have found that the width of the substrate blank region depends mainly on the height of convex and the water contact angle of highly-hydrophobic material or the absorptivity of water-absorbing material.

In one analysis-chip of the first embodiment or the third embodiment of the present invention, said highly-hydrophobic convex presents a height of 0.1-100 μm. In fact, in the examples, when its height is 20-50 μm, desirable results become obtainable.

In our research on analysis-chip, we have found that high-hydrophobicity can be used as an independent reactor-partition mechanism for limiting water solution movement on the surface of hydrophilic substrate surrounded by highly-hydrophobic material. Table 1 reveals the effects of some highly-hydrophobic coatings:

TABLE 1
Partition effects of some highly-hydrophobic coatings

| | Static | | | | | The | |
|---|---|---|---|---|---|---|---|
| | water | The | The | | Surface area | subjecting | Down |
| Highly-hydro- | contact | width of | thickness | | of hydrophilic | quantity | flow at |
| phobic paint | angle | coating | of coating | | substrate | of water | 90° |
| Organosilicon | 95° | 1 mm | 30-40 | μm | 16 mm² | 20 μl | − |
| paint | | | | | | | |
| Nano-textile | 155° | 1 mm | 400 | μm | 16 mm² | 30 μl | − |
| Nanometer | 160° | 1 mm | 20-30 | μm | 16 mm² | 30 μl | − |
| silica CDJ7 | | | | | | | |
| Black paint | 85° | 1 mm | <5 | μm | 16 mm² | 15 μl | − |
| (ChuangKePeng) | | | | | | | |

| | | | | | | |
|---|---|---|---|---|---|---|
| Substrate | 45° | — | — | 16 mm² | 10 μl | + |
| without coating | | | | | | |

In the table, “down-flow at 90°” means making the analysis-chip vertical to the horizontal plane then observing whether there is the flow of water. When water does flow downward, the result is positive (+), when there is no water flow, the result is negative (−).

In one analysis-chip of the first embodiment or the third embodiment of the present invention, said highly-hydrophobic material presents a water contact angle that is over 70° bigger than that of said substrate probe region.

In one analysis-chip of the first embodiment or the third embodiment of the present invention, said highly-hydrophobic material presents a water contact angle that is over 90° bigger than that of said substrate probe region.

In one analysis-chip of the first embodiment or the third embodiment of the present invention, said highly-hydrophobic material presents a water contact angle that is over 110° bigger than that of said substrate probe region.

In one analysis-chip of the first embodiment or the third embodiment of the present invention, said highly-hydrophobic material includes highly-hydrophobic organic material or/and highly-hydrophobic nano-material.

In one analysis-chip of the first embodiment or the third embodiment of the present invention, said highly-hydrophobic material includes one or more of the following materials:

1). highly-hydrophobic organosilicon and its derivatives;

2). highly-hydrophobic fluororesin and its derivatives;

3). highly-hydrophobic polymer; and

4). paint or/and solid substance containing highly-hydrophobic nano-particle. Said organosilicon materials are organosilicon polymer, copolymer based on organo-siloxane bond, or their highly-hydrophobic derivatives. Said fluororesins include: fluororesin oligomer, polytetrafluoroethylene (PTFE), copolymer fluororesin (PFEP), polytrifluorochloroethylene and polypolyvinylidene fluoride. Said highly-hydrophobic polymers include highly-hydrophobic polypropylene, polyacrylate and their derivatives. In the coating or/and solid substance with said highly-hydrophobic nanoparticle, said nanoparticles include one or more of the following nanoparticles whose diameter is 1 nm-100 nm: gold, vanadium, lead, silver, iron and oxide powders thereof, silica, titanic oxide, alumina powders and their derivatives.

In one analysis-chip of the first embodiment or the third embodiment of the present invention, said water-absorbing material includes one or more of the following:

1). natural water-absorbing material;

2). solid porous material of hydrophilic inorganic compound; and

3). water-absorbing material of synthetic polymer.

In one analysis-chip of the first embodiment or the third embodiment of the present invention, said water-absorbing material includes one or more of the following:

a). capillary-structure-containing paper product, cotton product, or/and sponge as well as their modified materials,

b). calcium salt;

c). water-absorbing materials based on cellulose or its derivative,

d). water-absorbing materials based on starch or its derivative,

e). water-absorbing materials based on synthetic resin as well as compound generated by grafting, blocking and copolymerizing, paper product, cotton product, sponge and its modifier, calcium salt, water-absorbing cellulose material, water-absorbing starch material, and water-absorbing synthetic resin produced by grafting, blocking and copolymerizing.

The fourth embodiment of the present invention provides an analysis-chip, which comprises one or more open non-flow reactors with minimal-height, wherein said reactor comprises at least probe-ligand, substrate probe region, open partition-structure with minimal height, and optionally, reactor-protecting structure, wherein said open partition-structure comprises: 1). substrate blank region with a width of 0.5-10 mm; and optionally, 2). hydrophobic convex with a height less than 1000 μm, optimally less than 500 μm relative to said substrate probe region, wherein: 1). said substrate blank region and substrate probe region are on the same plane of a same substrate, which presents a water-absorptivity less than 0.1 g/g and a water contact angle as 40-80°; and 2). said hydrophobic convex contains, at least on its partial surface, hydrophobic material, wherein said hydrophobic material presents a water contact angle of 55-80°.

In fact, the analysis-chip of the fourth embodiment is the analysis-chip with the blank partition structure of the present invention. Contrary to the visions of many people, we have found with amazement through repeated experiments that even without the partition convex, if there is a blank region whose width is 1-10 mm, under the optimal sample subjecting amount (e.g. 10 μm is subjected on 16 mm²) and reactive conditions (e.g. 30 minutes at 37° C.), no cross-contaminations occurs in the reaction. The residual reactant can be absorbed by using water-absorbing material or pipette or even washed out directly, and there is also no cross-contamination. In the analysis-chip with the blank partition structure of the present invention, an optional hydrophobic convex is not the main part, but usually is employed as the marker line or/and partition auxiliary.

In one analysis-chip of the first embodiment or the fourth embodiment of the present invention, said hydrophobic convex includes hydrophobic coating.

In one analysis-chip of the first embodiment or the fourth embodiment of the present invention, said hydrophobic coating includes colored hydrophobic line, or/and colored hydrophobic strip.

The fifth embodiment of the present invention provides an analysis-chip, which comprises one or more flow reactors with minimal-height, wherein said reactor comprises at least:

1). probe-ligand;

2). substrate probe region with a water-absorptivity less than 0.1 g/g and a water contact angle as 40-80°;

3). convex flow-path comprising high-hydrophilic convex with a width of 5-4000 μm and a height of 0.05-1000 μm, optimally 0.05-500 μm relative to said substrate probe region; and optionally,

4). reactor-protecting structure, wherein said high-hydrophilic convex contains, at least in its partial surface, highly-hydrophilic material, wherein said highly-hydrophilic material presents a water contact angle less than 40°. In one of the analysis-chip of the present invention, said highly-hydrophilic material presents a water contact angle less than 30°. Said highly-hydrophilic materials include highly-hydrophilic nanometer material (e.g. highly-hydrophilic nanometer material) and highly-hydrophilic polymer material (e.g. highly-hydrophilic polyacrylate acid paint).

In fact, the analysis-chip of the fifth embodiment is the analysis-chip with convex flow-path of the present invention, that can be connected to one or more of the following structures: sample well, reagent well, test well, waste liquid well, separator etc. Said reactor is a flow or non-flow reactor. The convex flow-path of the invented chip differs particularly from the micro-channel path of present chips. The flow-path of the latter needs to make channels, troughs or capping, and is a concave flow-path, whose manufacturing is complicated and the cost is expensive.

In one analysis-chip of the first embodiment or the fifth embodiment of the present invention, said convex flow-path refers to a coating with said highly-hydrophilic material.

In one analysis-chip of the first embodiment or the fifth embodiment of the present invention, said convex flow-path comprises hydrophobic convex or/and highly-hydrophobic convex as its partition structure, wherein said convex presents a height less than 1000 μm, optimally less than 500 μm.

In one analysis-chip of the first embodiment or the fifth embodiment of the present invention, said convex flow-path comprises immobilized separating-media, including electrophoresis media or chromatography media.

In one analysis-chip of the first embodiment or the fifth embodiment of the present invention, said reactor comprises also the open partition structure mentioned in the first embodiment of the present invention.

The sixth embodiment of the present invention provides an analysis-chip, which comprises one or more open reactors with minimal-height, wherein said reactor comprises at least probe-ligand, substrate probe region, marking-system convex, and optionally, reactor-protecting structure, wherein said marking-system convex refers to marker-containing convex that does not cover said probe-ligand.

In fact, the analysis-chip of the sixth embodiment is the analysis-chip with fixed marker. This invented convex of marking system can be in various geometric figures, such as dots, lines, strips, spheres etc.

In one analysis-chip of the first embodiment or the sixth embodiment of the present invention, said marking-system convex refers to convex of controlled marker-releasing system, wherein said controlled marker-releasing system comprises marker and presents a half-releasing-period of said marker of more than 10 seconds, optimally more than 30 seconds, wherein said marker comprises labeling reagent and marking-ligand, wherein:

1). said labeling reagent includes one or more of the following reagents: enzyme, fluorescent dyestuff, chemiluminescent catalyst, nonferrous metal or nonferrous metallic salt, dyestuff and paint; and

2). said ligand includes one or more of the following substances: antigen, antibody, biotin, drug ligand polypeptides, DNA, RNA and the fragments thereof.

One example of said controlled marker-releasing system is the programmable release system, in which the release velocity and the lag-time of marker are defined mainly by the selected material and structure of the system itself. For the marker in said convex of marking system, the labeling methods include chemiluminescent labeling, excitation luminescent labeling, non-selective light reflection labeling and selective light reflection labeling.

In the present invention, the term “half-releasing-period” refers to the time needed to release 50% of markers from controlled marker-releasing system into the reactor under the condition of labeling reaction. The convex with controlled marker-releasing system can be of various technical designs. For instance, it can be made controlled-releasing agent in the shape of a sphere or other solid figures containing liquid or solid markers; or a dot or line etc., which contains markers and controlled-releasing agent. In these cases, the marking convex has a height less than 1000 μm, which is fixed on the periphery of or inside probe array in the reactor to form probe/marking system arrays.

In one analysis-chip of the first embodiment or the sixth embodiment of the present invention, said controlled marker-releasing system comprises said marker and controlled-releasing agent. In the present invention, the term “controlled-releasing agent” refers to the agent that can control or take part in controlling the releasing speed of the marker and delay the releasing. Some examples of the controlled-releasing agent are: the diffusion-controlling agent that controls the marker releasing through controlling the change of the network density; the chemical reaction-controlling agent that controls the marker releasing through controlling degradation; solvent activation-controlled release agent that controls the marker releasing through controlling the activation process (e.g. dissolving); the controlled-releasing agent that speeds up release though using water-absorbing and swelling materials inside the membrane to make the membrane dilate and disintegrate etc. With the diffusion-controlling system, the marker is released according to the fluctuation of density of diffusion-controlling agent (e.g. polymer with moderate solvency and dilatability) in the network; with chemical reaction-controlling system, the marker is released when the controlling agent (e.g. degradable polymer) is degraded; with the activation-controlling system, the marker is released when the controlling agent (e.g. membrane) is activated (e.g. dissolved) by solvent. For example, in the diffusion-controlling system with markers evenly dispersed or embedded in polymer that has moderate water-solubility (controlled-releasing agent), the releasing speed of the marker is decided by the dissolving and swelling speed of the polymer, thereby the marker concentration can be adjusted to accommodate the requirements of different testing procedures. Taking another example with the chemical reaction-controlling system in which the marker is evenly dispersed or embedded in the degradable polymer (controlled-releasing agent), the marker-releasing velocity is actually zero when sample is subjected. While labeling, the marker is released due to the degradation of polymer with the help of degrading agent (e.g. enzyme). For still another example, in the activation-controlling system made up of markers and membrane (controlled-releasing agent), the release or delayed release of the marker is decided by the speed of membrane dissolution and dilation.

In one analysis-chip of the first embodiment or the sixth embodiment of the present invention, said marking convex presents a height less than 1000 μm, and is fixed around the array of said probe-ligand in said reactor or inside the array of said probe-ligand to form an array of probe-ligand and marking system.

In one analysis-chip of the first embodiment or the sixth embodiment of the present invention, said convex of controlled marker-releasing system includes mono-sandwiched or multi-sandwiched structure made up of said marker and controlled-releasing agent, wherein said sandwiched structure refers to structure where the concentration of said marker is higher inside than outside. In multi-sandwiched structure, one or more types of markers and one or more types of controlled-releasing agents could be included. For example, the first layer may comprise the marker; the second layer may comprise the structure of enhancing agent. Said release-controlling agent includes water-soluble organic compounds or organic compounds, which will disintegrate in water solution. The former includes natural water-soluble organics, partially synthetic water-soluble organics and synthetic water-soluble organic compound etc. Said organic compounds include one or more of the following materials: carbohydrates and the derivatives thereof, plant starch and modified starch, plant gum, animal gum, modified cellulose, polymer and condensate.

In one analysis-chip of the first embodiment or the sixth embodiment of the present invention, said controlled-releasing agent includes water-soluble organic compound or organic compound which will disintegrate in water solution.

In one analysis-chip of the first embodiment or the sixth embodiment of the present invention, said organic compound includes one or more of the following materials: carbohydrate and its derivatives thereof, plant starch and modified starch, plant glue, animal glue, modified cellulose, polymer and condensate.

In one analysis-chip of anyone of above embodiments of the present invention, said reactor-protecting structure comprises protective unit with a distance less than 1000 μm, optimally less than 500 μm from said substrate probe-region; wherein said protective unit closes at least partially said reactor structure when no sample is subjected, and it is irreversibly removed completely or partially when sample is to be subjected.

The seventh embodiment of the present invention provides an analysis-chip, which comprises one or more reactors with minimal height, wherein said reactor comprises: 1). easily-detachable substrate easily dismantled if needed, or/and 2). reactor-protecting structure with minimal-height, comprising protective unit, wherein: i). said height, a distance from substrate probe-region to bottom plane of said protective unit is less than 100 μm, optimally less than 500 μm; and ii). said protective unit closes at least partially said reactor structure when no sample is subjected, and it is irreversibly removed completely or partially when sample is to be subjected.

In fact, the analysis-chip of the seventh embodiment refers to the optionally-using analysis-chip of the present invention, in which a part of reactors are used for the analysis while other reactors of the chip rest in protected situation. The optionally-using analysis-chip includes two types: the detachable analysis-chip and openable analysis-chip. In the former, substrate of the chip can be easily dismantled so that only a part of the substrate with a part of the reactors are used, e.g. the activated glass, metals, plastic plates etc. that have undergone the processes of pre-cutting. In the latter, reactors are covered by enclosing units (e.g. the plastic plate with a removable area made by mechanic cutting, aluminous plastic film, and plastic film with or without removable parts, etc.).

In one analysis-chip of the first embodiment or the seventh embodiment of the present invention, said protecting unit includes one or more of the followings: organic film or/and plate, film or/and plate of metal-organic complex, and slide.

In one analysis-chip of the first embodiment or the seventh embodiment of the present invention, said protecting unit is connected with the reactor through one or more of the following reversible/ irreversible enclosing structures: thermal enclosing structure, chemical enclosing structure and reversible or irreversible adhesive layer.

In one analysis-chip of the first embodiment or the seventh embodiment of the present invention, said protecting unit is precut for the convenience of possible removal.

The eighth embodiment of the present invention provides an analysis-chip with a high density of reactors, wherein:

1). said density of reactors is more than 2 reactors/cm², optimally more than 3 reactors/cm² on at least one plane of a substrate; and

2). said reactor comprises partition structure with a height less than 1000 μm, optimally less than 500 μm.

In one analysis-chip of the eighth embodiment of the present invention, said density is more than 5 reactors/cm².

In one analysis-chip of the eighth embodiment of the present invention, said reactor refers to said reactor with minimal-height, which is that mentioned in anyone of analysis-chip of above embodiments of the present invention.

The present invention also involves the top or bottom units used in the analysis-chip of the first or second embodiments. The top or bottom units include the substrate probe region and immobilized probe therein, such as the top or bottom units that form the striped-capillary reaction-chamber. Said units contain the substrate probe region and probe, and part or whole chamber wall, part or whole inlet and outlet, part or whole enclosing structure (e.g. elastic coating and wetness-proof coating), as well as other structures of the analysis-chip (e.g. the fixing structure). The material of the top unit is selected from one of the following materials or their combinations: glass, silicon, metal oxide, metals and polymer material as well as their respective derivatives.

The ninth embodiment of the present invention provides a base-plate used for the analysis-chips of the present invention, comprising: 1). more than one of substrate probe regions; and 2). one or more of the following reactor structures: (1). the convex flow-path by Claim 1 or any claim from 22 to 26; or (2). the open partition structure by Claim 1 or any claim from 10 to 16.

The tenth embodiment of the present invention provides a qualitative or/and quantitative analysis method, including:

1). subjecting sample into reactor of said analysis-chip and taking probe-selective reaction and marking reaction respectively, wherein said analysis-chip is any analysis-chip of the present invention (from the first embodiment to eighth embodiment); and

2). washing the reactor and analyzing the result of the reaction.

The eleventh embodiment of the present invention provides a test device, which comprises cleaning unit, wherein said cleaning unit is used for cleaning up the residual in analysis-chip reactor when reaction is finished, wherein said cleaning is performed though absorption with water-absorbing material in said cleaning unit, wherein said water-absorbing material presents a water-absorptivity larger than 0.5 g/g.

The advantages of the analysis-chip of the first embodiment of the present invention are: simple and easy preparation, lower cost, reliable reactor-partition, homogenous distribution of reaction media, small consumption of reaction media, high sensitivity, convenient performance etc.

The advantages of the analysis-chip of the second embodiment of the present invention (the analysis-chip with striped capillary reaction-chamber) are: it overcomes not only the disadvantages of unequal distribution of media (e.g. existence of air bubble) on the chip, as well as the requirement of more sample and marker, but also the defects of complicity in capillary analysis-chip manufacture and high cost; hence it possesses the advantages of simple and easy manufacturing, high sensitivity, less consumption of reaction media, convenient performance etc.

The advantages of the analysis-chip of the third or fourth embodiment of the present invention (the analysis-chip with highly-hydrophobic partition structure, the analysis-chip with water-absorbing partition structure, the analysis-chip with highly-hydrophobic/water-absorbing complex partition structure, the analysis-chip with the blank region) are: the minimal height of the partition structure brings about the maximization of reactor numbers, the minimization of subjected-sample volume, or/and convenience in washing; moreover the laser confocal scanner or laser scanner can be used directly for scanning.

The advantages of the analysis-chip of the fifth embodiment of the present invention (the analysis-chip with convex flow-path) are: the flow speed can be controlled by selecting different convex material; the preparation is simple and the cost is low.

The advantages of the analysis-chip of the sixth embodiment of the present invention (the analysis-chip with fixed marker) are: with the controlled marker-releasing system, Hook Effect can be reduced; therefore the sensitivities of test and device are improved; by simplifying the operation procedures and mechanical transporting system, the efficiencies of test and test device are raised, and automatic test is made possible; under dried condition, marker will have much higher stability.

The advantages of the analysis-chip of the seventh embodiment of the present invention (optionally-using analysis-chip) in the present invention are: only part of reactors on the analysis-chip will be involved in accordance with the need, while others can be well preserved; thereby cost is reduced.

### BRIEF DESCRIPTION OF THE DRAWINGS

FIG. 1A is the schematic diagram of the invented analysis-chip with multiple striped-capillary reaction-chambers;

FIG. 1B is the sectional drawing (along a-a line) of the analysis-chip showed in FIG. 1A;

FIG. 2A-2E are the schematic diagram of different types of reactors and structures of the invented chips;

FIG. 3A-3B are the vertical view (downwards) of the bottom unit of the enclosing structure of the invented striped-capillary reaction-chambers;

FIGS. 3C and 3D are the vertical view (upwards) of the top unit.

1. Striped-capillary reaction-chamber

2. Reactor structure

3. Convex of marking system

4. Open partition structure

5. Top plane

6. Bottom plane

7. Substrate probe region

8. Closed partition structure

9. Inlet

10. Outlet

11. Top unit

12. Bottom unit

13. Inlet structure on the top plane

14. Outlet structure on the top plane

15. Fixing structure on the top plane

16. Seal structure on the top plane

Implementation Methods

Definition of Terms

In the present invention, the term “analysis-chip” or “chip” in short, refers to a test device for qualitative and/or quantitative analysis, wherein the result of the specific reaction between a small amount of probe-ligand and the target molecule in a sample can be identified in an addressable way. Analysis-chip includes, but not only includes, the “biochip”, “microarray”, “bioarray”. The analysis-chip includes micro-channeled analysis-chip (e.g. micro-channeled biochip) and microarray analysis-chip (e.g. biochip, microarray, bioarray etc.), but as known to all it doesn't include the available rapid test strip. The analysis-chip in the present invention contains a mono-reactor or multi-reactors with or without the marking system, in which the probe-ligand is immobilized on a substrate with a probe-spot density more than 10 spots/cm² and a probe-spot covering area not larger than 1 mm²/spot.

In the present invention, the term “test device” refers to articles used in the quantitative or/and qualitative analysis, which contain probe-ligand reacting with the target in a sample, e.g. instrument and consumable which contain probe-ligand, and kit containing probe-ligand and marking-ligand. Some examples of the test device are: analysis-chip, ELISA plate, affinity electrophoresis strip, affinity chromatography column, planar chromatography reagent strip, analysis-chip kit, ELISA plate kit, affinity electrophoresis kit, etc. The quantitative or/and qualitative analysis can be performed either in vitro, or in vivo.

In the present invention, the term “probe-ligand” refers to all active materials that can be immobilized on a solid support in an addressable way so that the target in a sample can be captured by probe-ligand, e.g. DNA, polypeptides, cells, tissues and bio-components etc.

In the present invention, the term “ligand” refers to the material that is used to: 1) capture its ligate through interaction (including affinity effect, ion exchange, oleophilic effect, etc.), and 2). bind with labeling material to generate marker. For example, it includes one or more of the following materials: antigens, antibodies, ligates, aptamer screened by Systematic Evolution of Ligands by Exponential Enrichment (SELEX), ligands, polypeptides, polysaccharides, coenzyme, cofactors, antibiotics, steroid, viruses, cells, biotin, avidin etc.

In the present invention, the term “labeling material” refers to materials generating or taking part in the formation of the detection signal, such as Rhodamin, Cy3, Cy5 etc, which are commonly used in the analysis-chip detection.

In the present invention, the term “substrate” refers to solid support where probe in an analysis-chip are immobilized.

In the present invention, the term “reaction room” refers to the place in a reactor where probe-ligand and target react with each other. The reaction room includes the reaction well and reaction-chamber. The reaction well is an open reaction room in which the probe array is open during the reaction. The reaction-chamber is an closed reaction room in which the probe array is closed in its above part during the reaction. The reaction well includes the substrate well and probe. The reaction-chamber includes the substrate chamber and probe. The substrate well and substrate chamber all include substrates and reactor partition structures.

In the present invention, the term “reactor” refers to the whole system consisting of the reaction room and all the connected structures therein. A reactor includes the borderline or partition structure, substrate, probe immobilized on the substrate and other connected structures (e.g. flow-path, inlet structure, outlet structure, the fixed marker, etc.). In the present invention, the biochip is defined as mono-reactor biochip (N=1) and multi-reactor biochip (N=2 or >2) according to reactor number N presented by the chip. The reactor is defined as a flow reactor and a non-flow reactor depending whether the added liquid media can flow directionally in the reactor during the detection. The biochip characterized by the flow reactor and non-flow reactor is defined as a flow analysis-chip and a non-flow analysis-chip respectively. The reactor is defined as the open reactor and non-open reactor respectively depending whether the probe array in the reactor is open in the whole course of detection. Accordingly the biochip characterized by these reactors is defined as the open analysis-chip or non-open chip, respectively. The reactor of said analysis-chip can simultaneously be featured with several types of reactors mentioned-above. In the present invention, this kind of reactor is defined according to all the properties it possesses. The analysis-chip characterized by this type of reactor is defined in the same way. For example: when a probe array is open with no covering from above during the detection, and liquid media added flow directionally in the reactor, this reactor is defined as an open flow reactor; the corresponding analysis-chip is defined as an open flow biochip; others are analogous of this.

In the present invention, the term “base-plate” refers to the intermediate used for the preparation of chip, which hasn't been set with the probe. It is based on the substrate and combined with or without other structures (e.g. the partition structure). It will form an analysis-chip after being fixed with the probe-ligand. There may be one or more substrate regions on a base-plate. A single base-plate refers to the one without the partition structure. In such a case, a base-plate is just a substrate, (for example the amino glass slide available in market). A base-plate with partition structure is referred to as the base-plate with more than one of substrate probe regions. In such a case, a base-plate is composed of the substrate and partition structure. Once the probe-ligand is immobilized in a substrate ligand region, a reactor is formed. The base-plate with multiple substrate probe regions thus can form an analysis-clip with multi-reactors.

In the present invention, the term “reactor structure” refers to any indispensable structure for a reactor besides the substrate and probe, such as the partition structure, flow-path structure etc.

In the present invention, the term “convex” refers to a protruding structure whose height is more than 0 in relation to the plane of the substrate probe region, e.g.

In the present invention, the term “reactor partition structure” refers to the structure that coating, adherend etc. can prevent cross-contamination among reactors at least in the course of sample subjectiong. It includes the structures used to segregate part or all of the reactor structures (e.g. reaction well, reaction-chamber, flow-path, inlet structure, outlet structure, etc.). The reactor partition structure includes open and closed partition structures. The open partition structure refers to the reactor partition structure without a cover when the analysis-chip is in use. Conversely, the closed partition structure refers to the reactor partition structure with a cover when the analysis-chip is in use.

In the present invention, the term “reactor flow-path” refers to the flow-path network in the reactor. The convex flow-path of the chips of the present invention refers to the flow-path higher than the substrate plane, which contains water-absorbing or/and highly-hydrophilic material.

In the present invention, the term “reactor separation structure” refers to the structure in the reactor of chip, which presents sample-separation function.

In the present invention, the term “marking system” refers to a system in the analysis-chip (including analysis-chip device and analysis-chip kit), which is used for marking the reaction in the reactor of the chip.

In the present invention, the term “reactor protecting system” refers to a system that maintains the reactor activity in storage and transporting process, and also keeps the unemployed reactors from being contacted by reaction media when other reactors are in use. The reactor protecting structure is different from the reactor partition structure.

In the present invention, the term “polypeptide” includes natural or synthetic proteins, protein fragments, synthetic peptides, etc. Polypeptide includes the common targets in immunoassay and the popular ligands in detection, e.g. antigens, antibodies, etc.

In the present invention, the term “nano-particle” refers to solid support particle with a size of less than 500 nm, optimally 1-100 nm, at least in one-dimension of its three-dimensions.

In the present invention, the term “chip with convex flow-path” refers to an analysis-chip comprising convex flow-path; the term “analysis-chip with highly-hydrophobic partition structure” refers to an analysis-chip containing the partition structure with the highly-hydrophobic convex; the term “analysis-chip with water-absorbing partition structure” refers to an analysis-chip containing the partition structure with the water-absorbing convex; the term “analysis-chip with blank-region partition structure” refers to an analysis-chip containing the partition structure with substrate blank region of width is 0.5-10 mm,and optionally, hydrophobic convex; the term “analysis-chip with highly-hydrophobic/water-absorbing partition structure” refers to an analysis-chip containing the partition structure with the highly-hydrophobic convex and the water-absorbing convex; the term “analysis-chip with fixed marker” refers to an analysis-chip containing fixed marker; the term “optionally-using analysis-chip” in the invention refers to an analysis-chip with multi-reactors, where only part of reactors are to be involved in the testing if needed, without interfering with other reactors.

In the present invention, the term “water contact angle” refers to the water contact angle on a surface. For a long time, it has been widely acknowledged that contact angle 0 of liquid drop on the surface of a solid substance can be taken as the quantitative indicator of some specific moisture capacity. If liquid is dispersed entirely on the surface and forms a membrane, then the contact angle 0 is zero. If certain angle is formed between the drop of liquid and the surface of substance, the surface is not considered wet. Glass is the most commonly used material as the substrate of solid support of chips, whose water contact angle is about 45°.

With the following embodiments, more detailed explanations of the invention will be given. However, it should be pointed out that these examples are only the individual cases concerning the implementation of the present invention. Professionals should learn more about other aspects of the implementation based on these examples.

The substrate in the embodiment of the present invention includes hydrophilic substrate and hydrophobic substrate. The former includes the epoxy glass slide and epoxy-group cover glass made by epoxidation. The latter is the glass slide painted black made by us. The glass slides are purchased from USA ESCO SCIENTIFIC Comp. The dimension is 76×26×1.0 mm; the cover glass is 60×24×0.15 mm. The probes purchased from Hepatic Disease Institute, Beijing People Hospital are HIV1+2 antigens, HBs antigens, HCV antigens, HBs antibodies respectively. The spotting concentration is within 1.0-1.5 mg/ml. In the embodiments, Sample 1 is HCV antibody-positive serum. Sample 2 is HIV1+2 antibody-positive human serum. Sample 3 is HBs antibody-positive human serum. Sample 4 is HBs antigen-positive human serum. Sample 5 is a negative control. All the samples are predetermined under the same testing condition by the classic open analysis-chip with the mono-reactor.

### IMPLEMENTATION EXAMPLE 1

### Preparation of the Analysis-Chip with Highly-Hydrophobic Partition Structure as well as the Application Thereof

In this embodiment, the liquid highly-hydrophobic materials in use are “polyacrylate paint” (made by China Chengdu Chenguang Research Institute of Chemical Industry, with a water contact angle as 85°), “organosilicon water-proof paint” (made by China Chengdu Chenguang Research Institute of Chemical Industry, with a water contact angle as 116°),“highly-hydrophobic latex paint” (supplied by China Chengdu Chenguang Research Institute of Chemical Industry, with a water contact angle as 123°), “highly-hydrophobic silica paint” (supplied by China Zhoushan Mingri Nanomaterials Co., Ltd., with a water contact angle as 151°) respectively. The solid highly-hydrophobic materials in use herein are “polytetrafluoroethylene pressure-sensitive adhesive tape” (supplied by China Chengdu Chenguang Research Institute of Chemical Industry, with a water contact angle as 117°), and “nanometer textile” (supplied by China Zhoushan Mingri Nanomaterials Co., Ltd., with a water contact angle as 155°) respectively. Although the reactor partition structure of the invented chip could partially or entirely be the device of the present invention, this embodiment only takes the simplest cases. Other cases may be illustrated in the following embodiments.

1) The Preparation of the Substrate with Highly-Hydrophobic Partition Structure

(1) Preparation of the substrate with highly-hydrophobic partition structure by solidifying the highly-hydrophobic liquid material. Coat said liquid highly-hydrophobic materials onto the surface region of the epoxy-group slide, where the partition structure will be formed. In accordance with the supplier's instruction, solidify it after it is dried at room temperature, form a highly-hydrophobic convex whose height is 25-115 μm and whose width is 2.0-2.5 mm. The convex can be made into various geometric shapes. In this embodiment, only two types are used: strip (the convex is in a shape of strip) and line combination (e.g. the convex has two lines with space in between). The surface enclosed by highly-hydrophobic convex could be in various geometric figures. In this embodiment, 3 mm×3 mm rectangle is used. On the surface of the substrate, there are 20 substrate-wells altogether, 10 substrate-wells being arranged lengthwise and 2 substrate-wells transversely.

(2) Preparation of the substrate with highly-hydrophobic partition structure by fixing the solid highly-hydrophobic materials Adhere the highly-hydrophobic solid material onto the surface region of the epoxy-group slide, where the partition structure will be formed. So, a highly-hydrophobic convex is formed on the substrate, whose height is 105-435 μm, and whose width is 2.0-2.5 mm. The surface enclosed by the highly-hydrophobic convex could be in various geometric shapes. In this embodiment, the surface enclosed by highly-hydrophobic convex is a 3 mm×3 mm rectangle. On the surface with highly-hydrophobic convex of the substrate, there are 56 substrate-wells altogether, 14 substrate-wells being arranged lengthwise, and 4 substrate-wells transversely.

2) The Preparation of the Analysis-Chip with Highly-Hydrophobic Partition Structure

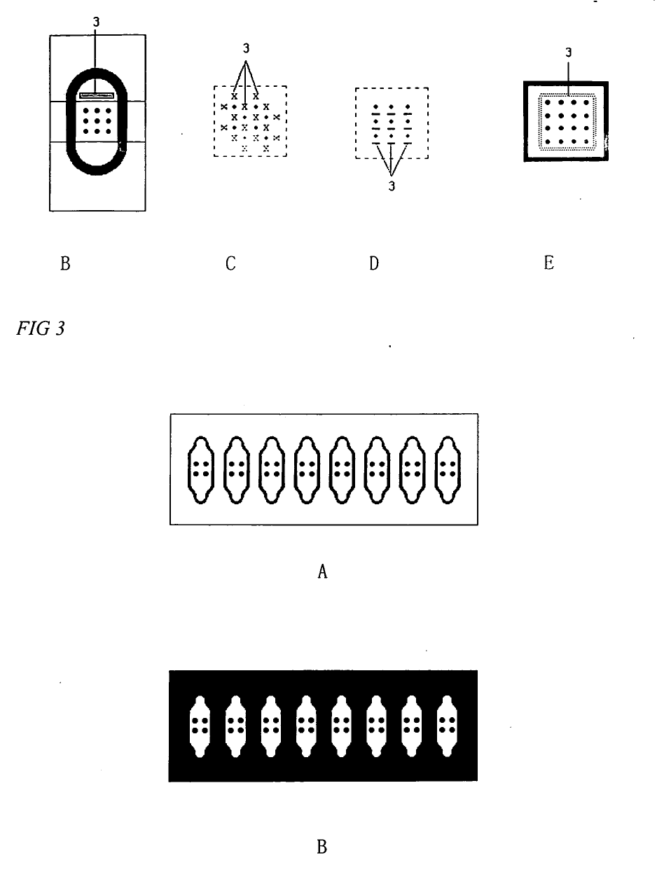

In fact, the invented analysis-chip can be prepared by two methods: (1). The probe immobilization before the formation of the highly-hydrophobic convex; and (2). The probe immobilization after the formation of the highly-hydrophobic convex. This embodiment adopts the second method. In the probe region 0.5 mm away from said highly-hydrophobic convex in the substrate-well, 3 types of antigens mentioned-above are immobilized by the known method of probe spotting. A 3×3 probe array thus comes into being, with each antigen for 3 spots. Then block the analysis-chip with bovine serum albumin. The final structure of reactor (FIG. 2A) includes the probe region on substrate and open partition structure.

3) Evaluation and Applications of the Analysis-Chip with Highly-Hydrophobic Partition Structure

In the experiment, take 10 chips of each type mentioned-above, number the reaction wells, any of the two reaction wells border upon are added respectively the positive serum of the mixture of the same volumes of Sample 1, 2, 3 and the negative control serum. The marker used is the mouse monoclonal antibody labeled with Rhodamine, which is self-made with the known method. In the experiment, the sample subjecting volume is 10 μl; the marker subjecting volume is 10 μl. When the sample is added and after the reaction is completed, there is no need to suck all the unbound substance in the reaction wells before washing as performed in ELISA. Instead, washing is conducted in accordance with one of the following approaches: (a) suck the unbound substances in the reaction well slightly with absorbent paper. And then rinse it for 1 minute with wash solution; (b) rotate the slide to form an angle of 45° with the horizontal plane, and then rinse it for 1 minute by spraying washing solution downward; (c) rotate the slide to form an angle of 180° with the horizontal plane, and then rinse it for 1 minute by spraying washing solution upward. The marker is added, washed, and dried with the known methods. After being dried, it is scanned with scanners. The scanner used herein is a laser confocal scanner (Afymetrix, GMS 418 microarray scanner), with wavelengths of excitation light at 532 nm; emission light at 570 nm. The visualized signal is processed by software (JAGUAR II). The cross-contamination rate is defined as follows: the numbers of reaction wells, in which the acquired results disagree with the added samples, is divided by total numbers of reaction wells examined. The experimental results are showed in Table 2:

TABLE 2

| | | | The | | | | Cross- |
|---|---|---|---|---|---|---|---|
| | Highly-hydrophobic | | thickness | | Convex | Blank | Contamination |
| Chip | material | Substrate | of convex | | structure | space | rate |
| 1 | Polyacrylate paint | Epoxy slide | 75-105 | μm | Strip | >500 μm | 0 |
| 2 | Organosilicon | Epoxy slide | 20-115 | μm | Strip | >500 μm | 0 |
| | water-proof paint | | | | | | |
| 3 | Organosilicon | Epoxy slide | 20-89 | μm | Strip | >500 μm | 0 |
| | water-proof paint | | | | | | |
| 4 | Organosilicon | Epoxy slide | 15-106 | μm | The line | >500 μm | 0 |
| | water-proof paint | | | | combination of | | |
| 5 | Highly-highly- | Epoxy slide | 75-105 | μm | Strip | >500 μm | 0 |
| | hydrophobic latex paint | | | | | | |
| 6 | Highly-hydrophobic | Epoxy slide | 65-89 | μm | Strip | >500 μm | 0 |
| | silica paint | | | | | | |
| 7 | Polytetrafluoroethylene | Epoxy slide | 120 | μm | Strip | >500 μm | 0 |
| | pressure-sensitive | | | | | | |
| | adhesive tape | | | | | | |
| 8 | Nanometer textile | Epoxy slide | 400 | μm | Strip | >500 μm | 0 |
| 9 | Nanometer textile | Black paint | 400 | μm | Strip | >500 μm | 0 |
| | | slide | | | | | |

### IMPLEMENTATION EXAMPLE 2

### Preparation of the Analysis-Chip with Water-Absorbing Partition Structure and Applications Thereof

In is embodiment, the water-absorbing materials in use are: “starch grafting acrylic acid water-absorbing membrane (whose thickness is 80-100 μm, water absorbability 450 g/g, provided by Chenguang Research Institute of Chemical Industry)”, “water-absorbing paper (whose thickness is 80-100 μm, water absorbability 150 g/g, provided by Chenguang Research Institute of Chemical Industry)” and deacetylated chitin grafting acrylonitrile water-absorbing membrane (whose thickness is 80-100 μm, water absorbability 220 g/g, provided by Chenguang Research Institute of Chemical Industry)” respectively. Although the partition structure of the invented reactor could be partially or entirely the device of the present invention, this embodiment only takes the simplest

1) Preparation of the Substrate with Water-Absorbing Partition Structure

First, punch 3 mm holes in said water-absorbing material, adhere the material onto the epoxy slide, and then form a water-absorbing convex with 3 mm upright holes whose height is 105-435 μm, and the space between the two holes is 2.0-2.5 mm. The convex can be in various geometric figures. In this embodiment, only strip figure is used (convex is in a shape of a strip). On the surface of the substrate, there are 20 wells (substrate-well) altogether, 10 wells in lengthwise direction and 2 wells in transverse direction.

2) Preparation of the Analysis-Chip with Water-Absorbing Partition Structure

The preparation method is the same as that of analysis-chip with highly-hydrophobic partition structure in Embodiment 1. The block treatment of the analysis-chip is performed with bovine serum albumin. Then, it is dried and made ready for use.

3) Evaluation and Applications of the Analysis-Chip with Water-Absorbing Partition Structure

In the experiment, take 10 chips of each type mentioned-above, number the reaction wells, any of the two reaction wells border upon are added respectively the positive serum of the mixture of the same volumes of Sample 1, 2, 3 and the negative control serum. The subjecting volume of sample is 7 μl; that of marker is 7 μl. The experimental method is the same as that in Embodiment 1. The cross-contamination rate is defined as follows: the numbers of reaction wells, in which the acquired results disagree with the added samples, is divided by total numbers of reaction wells examined. The cross-contamination rate on the analysis-chip prepared in this embodiment is zero.

### IMPLEMENTATION EXAMPLE 3

### Preparation of Analysis-Chip with a Blank Region as Partition Structure and the Applications Thereof

In this embodiment, the hydrophobic materials in use are: “black polyacrylate paint” (supplied by China Chengdu Chenguang Research Institute of Chemical Industry, with a water contact angle as 78°), “black paint” (supplied by China Chengdu Chenguang Research Institute of Chemical Industry, with a water contact angle as 76°). The substrate used herein is the epoxy glass slide. Although the partition structure of the invented reactor could be partially or entirely the device of the present invention, this embodiment only takes the simplest cases.

1) Preparation of the Substrate Containing the Marking Line with a Blank Region as Partition Structure

Coat the hydrophobic paint mentioned-above on the epoxy glass slide to form a black marking line whose height is 30-40 μm, and whose width is 500-1000 μm. On the surface of the substrate, there are 56 substrate-wells altogether, 14 in lengthwise direction, and 4 in transverse direction.

2) The Preparation of the Analysis-Chip with a Blank Region as Partition Structure

(1) The preparation of analysis-chip with a completely blank region as partition structure. The partition structure with a completely blank region doesn't contain hydrophobic convex. It is made by directly immobilizing several probe arrays onto the surface of the substrate. The immobilization of each probe array in this embodiment is the same as that in Embodiment 1. There are 10 probe arrays immobilized lengthwise and 2 probe arrays immobilized transversely, 20 probe arrays altogether. The substrate blank region between every two probe-arrays is 4.5 mm in width. The analysis-chip is blocked with bovine serum albumin and made ready for use.

(2) Preparation of the analysis-chip containing the marking line with a blank region as partition structure. Said preparation is based on the substrate prepared above, which contains the marking line with a blank region as partition structure. The preparing method is the same as that in Embodiment 1.

3) Evaluation and Applications of the Analysis-Chip with a Blank Region as Partition Structure

In the experiment, take 10 chips of each type mentioned-above, number the reaction wells, any of the two reaction wells border upon are added respectively the positive serum of the mixture of the same volumes of Sample 1, 2, 3 and the negative control serum. The subjecting volume of sample is 7 μl; that of marker is 7 μl. The experimental method is the same as that in Embodiment 1. The cross-contamination rate is defined as follows: the numbers of reaction wells, in which the acquired results disagree with the added samples, is divided by total numbers of reaction wells examined. The cross-contamination rate on the analysis-chip prepared in this embodiment is zero.

### IMPLEMENTATION EXAMPLE 4

### Preparation of the Analysis-Chip with Highly-Hydrophobic/Water-Absorbing Complex Partition Structure and the Applications Thereof

In this embodiment, the highly-hydrophobic liquid substance in use is “organosilicon water-proof paint” (supplied by China Chengdu Chenguang Research Institute of Chemical Industry, with a water contact angle as 116°); the water-absorbing material used herein is “starch grafting acrylic acid water-absorbing membrane (whose thickness is 80-100μm, water absorbability 450 g/g, provided by Chenguang Research Institute of Chemical Industry)”. Though the partition structure of invented reactor could be made up partially or entirely of the device of the invention, this embodiment only presents the simplest cases as the samples.

1) Preparation of the Substrate with Water-Absorbing Partition Structure

First, punch 3 mm holes in said water-absorbing solid, painting the said highly-hydrophobic paint with 0.5-1.0 mm width onto the said water-absorbing solid near the each hole, adhere the material onto the borderline of the reactors on the epoxy glass slide, and then form a water-absorbing convex with 3 mm upright holes whose height is 105-435 μm, and the space between the two holes is 2.0-2.5 mm. The convex can be in various geometric figures. In this embodiment, only strip figure is used (convex is in a shape of a strip). On the surface of the substrate, there are 20 wells (substrate-well) altogether, 10 wells in lengthwise direction, and 2 wells in transverse direction.

2) Preparation of the Analysis-Chip with Highly-Hydrophobic/Water-Absorbing Partition Structure

The preparing method is the same as that of the analysis-chip with highly-hydrophobic partition structure in Embodiment 1. The analysis-chip is blocked with bovine serum albumin, dried and then is made ready for use.

3) Evaluation and Applications of the Analysis-Chip with Highly-Hydrophobic/Water-Absorbing Partition Structure

In the experiment, take 10 chips of each type mentioned-above, number the reaction wells, any of the two reaction wells border upon are added respectively the positive serum of the mixture of the same volumes of sample 1, 2, 3 and the negative control serum. The subjecting volume of sample is 7 μl; that of marker is 7 μl . The experimental method is the same as that in Embodiment 1. The cross-contamination rate is defined as follows: the numbers of reaction wells, in which the acquired results disagree with the added samples, is divided by total numbers of reaction wells examined. The cross-contamination rate on the analysis-chip prepared in this embodiment is zero.

### IMPLEMENTATION EXAMPLE 5

### Preparation of the Analysis-Chip with the Controlled Marker-Releasing System and the Applications Thereof

The invented analysis-chip with the controlled marker-releasing system is actually an analysis-chip with a marker release-controlling system in the reactors. Similar to the drug release-controlling system, it also includes a programmable release system and an intelligent release system. The latter includes a release system with the external adjustment and intelligent gel. The marker release-controlling system of the present invention can be prepared the same way as the existing drug release-controlling system.

For the programmable drug release system, the methods of dissolving system can be prepared via using the hydrogel microsphere of ortho-hydroxybenzoic acid cross-linked by Ca²⁺ ion to embed drug, using the hollow body of polylactic acid to embed drug, using the hollow body of polylactic acid to embed foaming agent, using microsphere of water soluble polymers such as hydroxy-propyl cellulose or hydroxy-propyl methyl cellulose to embed drug, or using the intelligent drug release system with the external adjustments (e.g. through light, heat, PH value, electromagnetic, ultrasonic wave etc.).

Though this embodiment only offers the simplest cases for the preparation of the invented controlled marker-releasing system, it is clear to professionals that the method mentioned here can be used to prepare a variety of controlled marker-releasing systems.

1) The Half-Release Period of the Markers with Controlled Release System Switched on by Solvent

In this embodiment, the controlled marker-releasing system is composed of the controlled-releasing agent and membrane-enclosed powder marker formed by excipient. The marker used here is enzyme-labeled goat-anti-human secondary antibody (Beijing Tiantan Biological Products Co., Ltd.). The controlled-releasing agent used herein is the water-soluble organics (see Table 3) whose coating thickness is 80-100 μm. The controlled release system is switched on by dissolution with PBS buffer solution as a solvent. First, the solution of controlled-releasing agent with proper concentration will be coated onto the slide, forming a coating of 1 mm×1 mm×0.1 mm (length×width×height). After being dried, this coating is again painted with concentrated marker solution. After the marker is dried, the concentrated solution of controlled-releasing agent is coated to enclose the marker and is dried again for future use. At 37° C., a certain amount of solvent will be added to the controlled release system. The soluble parts will be taken out in different time. After that, half-release period can be calculated by detecting the dissolved marker through antibody-secondary antibody reaction. The half-release periods of marker in some controlled release systems switched on by solvent are showed in Table 3.

TABLE 3

| Controlled-releasing agent | Half-release period (minute) |
|---|---|
| Starch | 2 |
| Gelatin | 3 |
| Hydroxypropyl methyl cellulose | 5 |
| Hydroxypropyl cellulose | 12 |
| Acacia | 15 |

2) Preparation of the Analysis-Chip with the Controlled Marker-Releasing System

The marker in the marking convex on said analysis-chip of this embodiment is Rhodamine-labeled goat-anti-human secondary antibody. Acacia and ethyl cellulose are used as controlled-releasing agents. The substrate used herein is the substrate of analysis-chip with highly-hydrophobic partition structure based on epoxy glass slide prepared in Embodiment 1.

(1) Fixing the Marking Convex in the Periphery of the Probe Array in the Reactor

In embodiment 1, said three antigen probes are immobilized in the substrate well to form a probe array, which is then blocked with bovine serum albumin solution. Then Acacia with proper concentration is painted around bottom of the reaction well, forming a 1 mm-wide strip. After being dried, the marker containing starch as excipient is coated onto the strip of Acacia, which is dried again and coated with Acacia for enclosing. After it is dried, it is made ready for use. The marking convex prepared in this way has a thickness of 350-480 μm. Thereby in the prepared reactor structure (FIG. 2E), the marking convex 3 is outside the substrate probe region with the open partition structure around it. Of course, the marking convex can also be surrounded by the closed partition structure (FIG. 2B). And the half-release period of the marker at 37° C. is tested for 15 minutes.

(2) Fixing the Marking Convex Inside the Probe Region to Form the Probe/Marking System Arrays

Following the probe-spotting method in embodiment 1, the marker solution containing gelatin (secondary antibody 1 mg/ml) at a proper concentration and probe solutions of three antigens are spotted respectively into the substrate-well of the substrate to form the probe/marker arrays. The reactor (FIGS. 2C, 2D) thus obtained has the array of probe/marking convex 3 inside the substrate probe region, surrounded with the open partition structure. The half-release period of the controlled release system at 37° C. is tested for about 3 minutes.

3) The Application of the Analysis-Chip with the Controlled Marker-Releasing System

In experiment, four samples are subjected into said analysis-chip with the controlled marker-releasing system respectively, with each sample in 2 reaction wells. The sample subjected should be diluted properly. In this embodiment, 5 μl of samples should be subjected and kept under incubation at 37° C. for 5 minutes. After that, washing should be conducted for 3 times, with 15 μl of washing solution added for each washing. And then, after 15 μl of diluent is subjected, there is another 5-minute incubation at 37° C. After reaction, it is rinsed 5 times with 15 μl of washing solution added each time. Scanning (with GMS 418 microarray scanner made by Afymetrix,) will be conducted after drying. The processed data are listed in Table 4.

TABLE 4

| | | | Controlled |
|---|---|---|---|
| | | | marker |
| | | Standard | releasing |
| Method | | ELISA method | method |
| HCV antibody-positive | HCV antibody | + | + |
| serum | HIV antibody | − | − |
| HIV antibody-positive | HCV antibody | − | − |
| serum | HIV antibody | + | + |
| Positive control | HCV antibody | + | + |
| | HIV antibody | + | + |
| Negative control | HCV antibody | − | − |
| | HIV antibody | − | − |

“+” stands for the positive result; “−” the negative result.

### IMPLEMENTATION EXAMPLE 6

### Preparation of the Analysis-Chip with Convex Flow-Path and the Applications Thereof

This embodiment illustrates how a micro-channel path is prepared with highly-hydrophilic material. The invented analysis-chip with highly-hydrophilic micro-channel path differs from the present micro-channel path analysis-chip mainly in that the former employs the surface of highly-hydrophilic material as an important component for micro-channel path.

Though there are many highly-hydrophilic materials with varied hydrophilicities available for selection, this embodiment illustrates only two materials. The highly-hydrophilic liquid material used herein is “highly-hydrophilic nanometer silica paint” (provided by China Zhoushan Mingri Nanomaterials Co., Ltd., with a water contact angle as 28°) and “highly-hydrophilic polyacrylic acid paint” (supplied by China Chengdu Chenguang Research Institute of Chemical Industry, with a water contact angle as 35°).

1) Preparation of the Chip with Highly-Hydrophilic Micro-Channel Path which Does not Present Micro-Channel Partition Structure

The highly-hydrophilic liquid material is used to draw a line with a width of 80-100 μm and with a length of 10 mm on the surface of the slide. After being dried, it is ready for use.

2) Preparation of the Chip with Highly-Hydrophilic Micro-Channel Path which Presents Micro-Channel Partition Structure

“Organosilicon water-proof paint” (supplied by China Chengdu Chenguang Research Institute of Chemical Industry whose water contact angle is 116°) is coated onto both sides of said highly-hydrophilic micro-channel path without the partition structure, with a width of 1 mm. After being dried, the highly-hydrophilic micro-channel path with the partition structure is ready.

3) Applications of the Highly-Hydrophilic Micro-Channel Path

Set said slide horizontally. Subject 10 μl of PBS buffer solution into one end of the prepared highly-hydrophilic micro-channel path. As a result, a phenomenon similar to capillarity occurs instantly, that is, the liquid gradually flows from the liquid-subjecting end to the other end.

It is easy for professionals to apply the invented micro-channel path to the analysis-chip with micro-channel path, such as PCR chip, micro-extraction chip, chemical synthetic chip, cell counting chip, integrated analysis-chip with multi-system, bio-microarray, multi-channeled integrated chip, capillary electrophoresis chip, lab-on-chip, etc.

### IMPLEMENTATION EXAMPLE 7

### Preparation of Optionally-Using Analysis-Chip and the Applications Thereof

The analysis-chip prepared in this embodiment is the optionally-using analysis-chip, including two types: the detachable analysis-chip in which selected reactors can be detached,and openable analysis-clip in which selected reactors can be opened, according to the need of the analysis. In the former, substrate can be easily dismantled; while in the latter, reactor is covered by sealing unit.

1) Preparation of the Detachable Chip

The easily-detachable substrate used in this embodiment is a polystyrene plate with a precut ditch that can help dismantle the substrate if needed. The area surrounded by the precut ditch is 4×4 mm. The ditch is 1 mm wide and 0.8 mm deep. Then “Organosilicon water-proof paint” (supplied by China Chengdu Chenguang Research Institute of Chemical Industry, with a water contact angle as 116°) is painted onto the border of the area surrounded by the precut ditch, forming a highly-hydrophobic convex line with a height of 80-100 μm and a width of about 1 mm. Thereby a substrate-well comes into being. After that, said HCV fusion antigen, HIV1+2 fusion antigen, and HBs antigen are spotted respectively in 3 spots into each substrate-well, forming a 3×3 arrays; while the areas on the substrate without being spotted will be blocked and dried for future use. The same preparation method is applicable to other detachable chips with an easily-detachable substrate (e.g. the activated glass slide, metal plates, plastic plates etc., which have undergone the pre-cutting).

2) Preparation of the Openable Chip

The reactor-sealing unit used in this embodiment includes: the plastic plate with a removable area made by mechanic cutting, aluminous plastic film, and plastic film with or without removable parts, etc.

Spotting is conducted in the same way as mentioned above on the substrate prepared in Embodiment 3, which contains a marking line, with a blank region as the partition structure. After 20 reaction wells are formed on the substrate, waterproof pressure-sensitive adhesive with a height of 80-100 μm, and a width of about 1 mm is coated onto the border of the reactor well, which will then be enclosed by reactor-sealing units.

3) Application of the Optionally-Using Analysis-Chip

In testing, different number of reaction wells can be taken as required by dismantling parts of the substrate in the detachable analysis-chip along the precut ditch or by uncovering the easily-removable part of said openable analysis-chip with vacuum pumps, or removing the covers above the reaction well by mechanic cutting while keeping other reaction wells intact. In the experiment, take 10 chips of each type mentioned-above, number the reaction wells, any of the two reaction wells border upon are added respectively the positive serum of the mixture of the same volumes of sample 1, 2, 3 and the negative control serum. The subjecting volume of sample is 7 μl; that of marker is 7 μl. The experimental method is the same as that in Embodiment 1. The cross-contamination rate is defined as follows: the numbers of reaction wells, in which the acquired results disagree with the added samples, is divided by total numbers of reaction wells examined. The cross-contamination rate on the analysis-chip prepared in this embodiment is zero.

### IMPLEMENTATION EXAMPLE 8

### Application of the Analysis-Chip with High Density of Reactors and the Applications Thereof

1) Preparation of the Analysis-Chip with High Density of Reactors

In the embodiment, the prepared analysis-chip with high density of reactors uses epoxy glass slide as the substrate. Hydrophobic liquid material “polyacrylate paint” (supplied by China Chengdu Chenguang Research Institute of Chemical Industry, with a water contact angle as 85°) is coated onto the substrate to form the highly-hydrophobic convex with a height about 100 μm, and a width about 1 mm, and form a substrate-well of 2×2 mm. After that, 3 types of antigen solutions will be deposited with an arrayer (GM417ARRAYER, GENETIC MICROSYSTEM COMP.) into said reserved area in the pattern of 10 spots for each type of ligand, so as to form 5×6 probe arrays. The probe array (the substrate probe region) is 1×1 mm in size. The density of reaction well on the substrate is over 9 units/cm².

2) Evaluation and Application of the Analysis-Chip with High Density of Reactors

In the experiment, take 10 chips mentioned-above, number the reaction wells, any of the two reaction wells border upon are added respectively the positive serum of the mixture of the same volumes of Sample 1, 2, 3 and the negative control serum. The subjecting volume of sample is 5 μl; that of marker is 7 μl. The experimental method is the same as that in Embodiment 1. The cross-contamination rate is defined as follows: the numbers of reaction wells, in which the acquired results disagree with the added samples, is divided by total numbers of reaction wells examined. The cross-contamination rate on the analysis-chip prepared in this embodiment is zero.

### IMPLEMENTATION EXAMPLE 9

### Preparation of Analysis-Chip with Striped Capillary Reaction-Chamber and the Application Thereof

1) Preparation of the Monoplane Analysis-Chip with Striped Capillary Reaction-Chamber

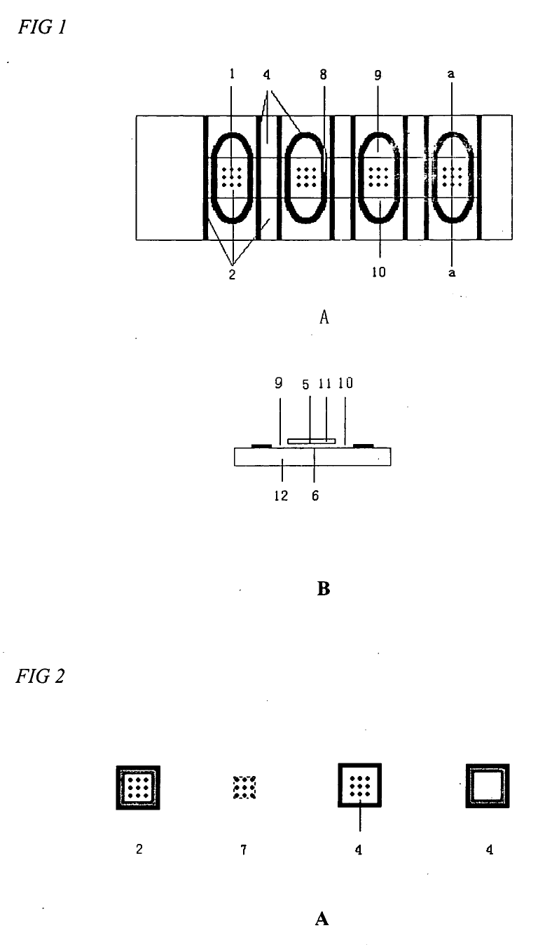

The monoplane analysis-chip with striped capillary reaction-chamber without the protection unit prepared in the embodiment is marked as A1 (as showed in FIG. 1). The monoplane analysis-chip with striped capillary reaction-chamber containing the protection unit is marked as A2.

The unit containing probes is prepared as below: on this substrate, “organosilicon highly-hydrophobic paint” (Chengdu Chenguang Research Institute of Chemical Industry) is coated to form a partition strip on the scheduled 8 inlet and outlet areas as shown in FIG. 1. The width of each substrate-well is 4 mm. After it is dried overnight, deposit above HCV fusion antigen solution and HIV fusion antigen solution in the pattern of 3 spots per each ligand into the above scheduled areas with an arrayer (GM417ARRAYER, GENETIC MICROSYSTEM COMP.), forming 3×3 probe arrays. After 3 hours of coating reaction at 37° C., it is blocked with bovine serum, rinsed and dried for use.

The formation of the striped-capillary reaction-chamber: the cover glass (60×12.5×0.15 mm in dimension) is used as the top unit 11; the unit with probes prepared as above is used as the bottom unit 12. The top unit 11 is connected to the bottom unit 12 through adhesion. The used adhesive is dual epoxy resin (omnipotent adhesive, Chengdu Chenguang Research Institute of Chemical Industry). In accordance with the instruction of product, spread the adhesive onto the highly-hydrophobic coating of the unit containing substrates. And then adhere it to the other unit. Of the prepared analysis-chip with striped capillary reaction-chamber, the width of its striped-capillary reaction-chamber 1 is 4000 μm. The space between the top plane 5 and bottom plane 6 is 80 μm. There is a closed partition structure 8 in the parts of the top plane and bottom plane among the striped-capillary reaction-chambers. There are the open partition structure 4, inlet 9, outlet 10 in the parts of liquid inlet and outlet. The water contact angle of the glass slide is 44°.

Preparation of the monoplane analysis-chip with striped capillary reaction-chamber containing the protection structure: the inlet and outlet area mentioned-above can be closed or opened according to the need by using the preparation method for the dismantled analysis-chip in Embodiment 7.

2) Preparation of the Double-Plane Analysis-Chip with Striped Capillary Reaction-Chamber

The double-plane analysis-chip with striped capillary reaction-chamber without the protection unit prepared in this embodiment is marked as A3 (as showed in FIG. 1). The double-plane analysis-chip with striped capillary reaction-chamber containing the protection unit is marked as A4.

The top unit is based on the amino-group cover glass. The bottom unit is based on the amino-group slide. Both top plane and bottom plane are immobilized with probes. By using the ligand solution mentioned-above, HCV and HIV antigens are deposited respectively in the same pattern onto the corresponding positions of the top and bottom units with an arrayer (GM417ARRAYER, GENETIC MICROSYSTEM COMP.), 2 spots each, forming 2×2 ligand arrays. After the coating reaction, it is sealed, rinsed and dried. The connection of the top and bottom units as well as the formation of the inlet and outlet is the same as in Embodiment 1). Of the prepared analysis-chip with striped capillary reaction-chamber, the dimensions of the top plane and bottom plane of the striped-capillary reaction-chamber is 4 mm×12.5 mm (width×height). The space between the top plane and bottom plane is 0.08 mm. The water contact angle of the glass slide is 44°.

3) Examination of Characteristics of the Analysis-Chip with Striped Capillary Reaction-Chamber

PBS is used as the test media in the embodiment. Set the analysis-chip obtained in 1) and 2) upright (inlet on the bottom, and outlet on the top). Deposit 5 ul of PBS with an 8-tipped pipette into the entrance of the striped-capillary reaction-chamber in the inlet well, and observe PBS solution filling the striped-capillary reaction-chamber through the inlet into outlet.

4) Utilization of the Analysis-Chip with Striped Capillary Reaction-Chamber

Sample 1, 2, and 5 are used in the embodiment. Before the samples are subjected, they are diluted properly. There are two approaches for subjecting sample:

(1) Subjecting sample by batches: the volume subject is 3 ul. During the processing, when the sample is subjected into the feeding well, the sample will fill the whole striped-capillary reaction-chamber from the inlet to outlet automatically due to capillarity There isn't air bubbles observed. Put the analysis-chip into an incubator for reaction at 37° C. for 5 minutes.

(2) Subjecting sample continually: during the processing, the sample is warmed up to 37° C. Subject the sample into the feeding well with the flow velocity of 1 ul/min. The subject volume is 10 ul. The subject time is 10 minutes. The sample will fill the whole striped-capillary reaction-chamber from the inlet to outlet owing to the capillarity. There isn't air bubbles observed. The reaction temperature is 37° C. The reaction time is 5 minutes.

After the reaction, the sample is absorbed with paper or drawn out by the pipette via the outlet. Washing solution could be subjected by batches or continually with the total volume of 20 ul.

The marker is Rhodamine-labeled goat anti-human secondary antibody (Jackson Immunoresearch Laboratories). In order to use the marker as few as possible, the batch-subjecting approach is applied. The subject volume is 3 ul, the reaction temperature is 37° C. and the reaction time is 5 minutes. Washing after the labeling reaction is the same as that for the sample reaction. After being dried, the results are visualized directly by using the laser confocal microscope scanner (Afymetric Comp, GMS 418 analysis-chip Scanner). The data are processed to get results showed in Table 6.

TABLE 6

| | HCV antibody | | HIV antibody | | | | | |
|---|---|---|---|---|---|---|---|---|
| | positive serum | | positive serum | | Positive control | | Negative control | |
| | HCV | HIV | HCV | HIV | HCV | HIV | HCV | HIV |
| Chip | antibody | antibody | antibody | antibody | antibody | antibody | antibody | antibody |

Subject sample by batches

| | | | | | | | | |
|---|---|---|---|---|---|---|---|---|
| A1 | + | − | − | + | + | + | − | − |
| * | | | | | | | | |
| A2 | + | − | − | + | + | + | − | − |
| * | | | | | | | | |
| A3 | + | − | − | + | + | + | − | − |
| * | | | | | | | | |
| A4 | + | − | − | + | + | + | − | − |
| * | | | | | | | | |
| A4 | + | − | − | + | + | + | − | − |
| ** | | | | | | | | |

Subject sample continually

| | | | | | | | | |
|---|---|---|---|---|---|---|---|---|
| A1 | + | − | − | + | + | + | − | − |
| * | | | | | | | | |
| A2 | + | − | − | + | + | + | − | − |
| * | | | | | | | | |
| A3 | + | − | − | + | + | + | − | − |
| * | | | | | | | | |
| A4 | + | − | − | + | + | + | − | − |
| * | | | | | | | | |
| A4 | + | − | − | + | + | + | − | − |
| ** | | | | | | | | |

“+” stands for the positive result; “−” is the negative result
“*” and “**” are the results of samples of 10-fold-diluted and of 20-fold-diluted respectively.

### IMPLEMENTATION EXAMPLE 10

### Application of the Analysis-Chip with the Irreversible closed Striped-Capillary Reaction-Chamber and the Application Thereof

The substrate (amino-group slide) and two probes (HCV antigen and HIV antigen) in the embodiment are the same as those in Embodiment 1.

1) Preparation of the Bottom Unit

(1) The material of the Wall and Seal Structure of the Striped-Capillary Reaction-Chamber of Bottom Unit are Elastic Material

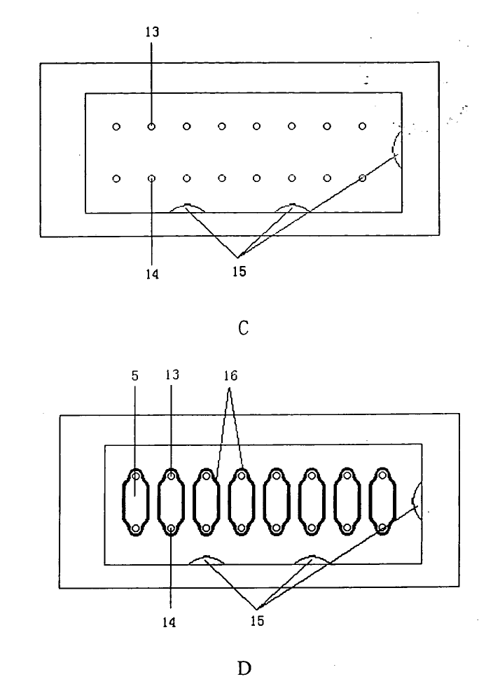

On the amino-group slide, elastic material (air-drying silicone rubber solution, Chengdu Chenguang Research Institute of Chemical Industry) is used to coat the whole areas evenly except 8 areas (refer to FIG. 3B; each area has a width of 4 mm) that are scheduled for fixing ligands. The thickness of elastic layer is less than 0.06 mm. After it is dried overnight, deposit the above ligands solution, in the pattern of 3 spots per ligand, into the above-scheduled areas with an arrayer (GM417ARRAYER, GENETIC MICROSYSTEM COMP.), forming 3×3 probe arrays. After 3-hour coating reaction at room temperature, it is blocked with bovine serum, cleaned and dried for use. The obtained bottom unit is marked as A1.

(2) The use of Highly-Hydrophobic Material for the Bottom Unit of the Wall and Enclosing Structure of the Striped-Capillary Reaction-Chamber

In the embodiment, the preparation of bottom unit is same as the unit-containing substrate in item 1) of Embodiment 1. The thickness of highly-hydrophobic material layer is less than 0.06 mm. The obtained bottom units that use amino glass slide and black paint glass slide are marked as A2 and A3 respectively

(3) The use of Highly-Hydrophobic and Elastic Materials Respectively for the Bottom Unit of the Wall and Seal Structure of the Striped-Capillary Reaction-Chamber

On an amino-group slide, the highly-hydrophobic material (organosilicon highly-hydrophobic paint, Chengdu Chenguang Research Institute of Chemical Industry) is coated onto the border of the 8 areas (as showed in FIG. 3A, each area has a width of 4 mm) scheduled for fixing ligand to generate a borderline layer. After it is dried, elastic material (air-drying silicone rubber solution, Chengdu Chenguang Research Institute of Chemical Industry) is coated onto the areas outside the highly-hydrophobic borders, forming a strip whose width is 2 mm. The layer of the elastic coating is a little higher than the highly-hydrophobic layer (The thickness of layer is less than 0.05 mm). And then it is coated with the ligand solution mentioned-above. The coating method is the same as (1) in 1) of this embodiment. The obtained bottom unit is marked as A4.

(4) The Material of the Wall and the Enclosing Structure of Striped-Capillary Reaction-Chamber of Bottom Unit is Adhesive Material

On amino-group slide, adhesive material (water-insoluble adhesive tape using polytetrafluoroethylene as substrate, Chengdu ChenGuang Research Institute of Chemical Industry) is used to adhere the areas outside 8 areas scheduled for fixing ligands (as showed in FIG. 3B, each area with a width of 4 mm) homogenously (the thickness of layer is less than 0.2 mm). And then it is spotted with ligand solutions mentioned-above. The spotted method is the same as (1) in 1) of the embodiment. The obtained bottom unit is marked as A5.

(5) The Bottom Unit Containing the Striped-Capillary Reaction-Chamber Without the Seal Structure

The ligands mentioned-above are coated onto the amino glass slide directly. The coating method is the same as (1) in 1) of the embodiment. The obtained bottom unit is marked as A6.

2) Preparation of the Top Unit

(1) Top Unit Without the Formation Structure

The top unit (See FIG. 3C) is made from stainless steel plate which can be used repeatedly, with a size as 100 mm×40 mm×2 mm (length×width×thickness) and has a inlet 13, and liquid-outlet 14, and liquid-in and out pipes, and a fixing structure 15. Its surface contacted with the bottom unit is a plane without any seal structure. Every pair of inlet and outlet is corresponding to the inlet and outlet areas of each reaction well on the bottom unit. The obtained top unit is marked as B1.

(2) The Top Unit with the Formation Structure

The top unit (FIG. 3D) is made from stainless steel plate which can be used repeatedly, with a size as 100 mm×40 mm×2 mm (length×width×thickness) and has a inlet and outlet 13 and liquid-in and -out pipes. On its plane contacted with the bottom unit there is the seal structure 16. Its seal structure is an elastic material layer (room temperature self-drying silicone rubber solution, Chengdu Chenguang Research Institute of Chemical Industry) (with a thickness less than 0.1 mm) corresponding to the areas outside the reaction wells on the bottom unit. Every pair of inlet and outlet is corresponding to the inlet area and outlet area of each reaction well on the bottom unit. The obtained top unit is marked as B2.

3) Examination of Characteristics of the Analysis-Chip with Striped-Capillary Reaction-Chamber

PBS is used as a test media in the embodiment. The top and bottom units of the analysis-chip obtained in 1) and 2) mentioned-above are connected with hermetization. This embodiment uses mechanic pressure to complete said seal connection. According to different combinations of the top and bottom units, varied chips are generated (See Table 7). Set the combined object of the top and bottom units upright (inlet on the bottom, outlet on the top). Subject PBS into the entrance of the striped-capillary reaction-chamber. Open the inlet and it can be observed that PBS solution is filling the striped-capillary reaction-chamber through the inlet to the outlet.

4) Use of the Analysis-Chip with Striped Capillary Reaction-Chamber Samples used in the embodiment are the same as those in Embodiment 1. During experiment, 4 samples are subjected into the analysis-chip (Table 7) with the striped-capillary reaction-chamber respectively. Each type of sample is subjected into 2 reaction wells respectively. Before the samples are subjected, they are diluted properly. Two approaches to subjecting sample:

(1) Subjecting Sample by Batches:

During operation, sample is subjected into the feeding well and is stopped when it reaches outlet. It will fill the whole striped-capillary reaction-chamber due to capillarity from the inlet to the outlet automatically There isn't air bubbles observed. Put the analysis-chip into an incubator for reaction for 5 minutes at 37° C.

(2) Subjecting Sample Continually:

During operation, sample is warmed up to 37° C. Subject the sample into the feeding well with the flow velocity of 10 ul/min at first and 1 ul/min later on. The subjecting time is 5 minutes.

After the reaction, the sample is absorbed with paper or drawn out by a pipette via the outlet. Washing solution could be subjected by batches or continually. The total volume subjected is 40 ul.

The marker is Rhodamine-labeled goat anti-human secondary antibody (Jackson ImmunoResearch Laboratories). In order to use the label volume as lease as possible, this embodiment uses the batched approach to subject sample. The subject volume is 5 ul. Reaction temperature is 37° C. Time for reaction is 5 minutes. The washing after the labeling reaction is the same as that for the sample reaction s. After it is dried, the results are visualized directly with a laser confocal microscope scanner (Afymetric Comp, GMS 418 analysis-chip Scanner). The data are processed to get the results showed in Table 7.

TABLE 7

| | | | HCV antibody | | HIV antibody | | | | | |
|---|---|---|---|---|---|---|---|---|---|---|
| | | | positive serum | | positive serum | | Positive control | | Negative control | |
| | Top | Bottom | HCV | HIV | HCV | HIV | HCV | HIV | HCV | HIV |
| Chip | unit | unit | antibody | antibody | antibody | antibody | antibody | antibody | antibody | antibody |
| 1 | B1 | A1 | + | − | − | + | + | + | − | − |
| 2 | B1 | A2 | + | − | − | + | + | + | − | − |
| 3 | B1 | A3 | + | − | − | + | + | + | − | − |
| 4 | B1 | A4 | + | − | − | + | + | + | − | − |
| 5 | B1 | A5 | + | − | − | + | + | + | − | − |
| 6 | B2 | A6 | + | − | − | + | + | + | − | − |
| 7 | B2 | A1 | + | − | − | + | + | + | − | − |
| 8 | B2 | A2 | + | − | − | + | + | + | − | − |
| 9 | B2 | A3 | + | − | − | + | + | + | − | − |

“+” stands for positive result; “−” negative result

### IMPLEMENTATION EXAMPLE 11

### Preparation of the Analysis-Chip with Ligand-Ligate and the Applications Thereof

1) Preparation of Chip with Ligand-Ligate

The substrate in the embodiment is the epoxy glass slide. Two probes are HBs fusion antigen and anti-HBs antibody (Hepatic Disease Institute, Beijing People Hospital).

The preparation of the monoplane analysis-chip with striped capillary reaction-chamber is the same as that in Embodiment 9. Though ligand and ligate in the invention could be in varied fashions and immobilized onto different corresponding positions, in the embodiment, the 3×3 arrays of HBs fusion antigen are immobilized near the inlet of each striped-capillary reaction-chamber; however, a 3×3 arrays of anti-HBs antibody are immobilized near the outlet.

2) Examination of Characteristics of the Capillary Chip

The method is the same as that in Embodiment 9

3) Application of the Chip with Ligand-Ligate

The samples used in this embodiment are HBs antigen positive human serum, HBs antigen negative human serum and HBs antibody positive human serum. All the samples are identified by a pre-analysis with the classical analysis-chip with the open mono-reactor under the same reactive condition. During experiment, 3 samples are placed into the prepared analysis-chip mentioned-above respectively. This embodiment uses the batch-approach subject sample in Embodiment 9. The test method is the same as that in Embodiment 9. The data are processed to get the results showed in Table 8.

TABLE 8

| Multiple of | HBs antigen | HBs antigen | HBs antibody |
|---|---|---|---|
| sample dilution | positive serum | negative serum | positive serum |
| 10 | + | − | + |
| 100 | + | − | + |

“+” stands for positive result; “−” negative result.

1-61. (canceled)

62. An analysis-chip, which comprises one or more reactors with minimal height, wherein said reactor comprises at least:
1). one or more striped-capillary reaction-chambers, wherein said reaction-chamber comprises:
i). top plane and bottom plane with a width more than 600 μm;
ii). probe-ligand and substrate probe-region in which said probe-ligand is immobilized;
iii). closed partition-structure with a height of 1-1000 μm, optimally 1-500 μm; and
iv). inlet and outlet,
2). reactor structure with minimal-height; which comprises at least open partition-structure comprising:
i). substrate blank region with a width of 0.5-10 mm; or/and
ii). one or more following convexes with a height less than 1000 μm, optimally less than 500 μm: hydrophobic convex, highly-hydrophobic convex, and water-absorbing convex; and optionally, iii). convex flow-path comprising high-hydrophilic convex with a width of 5-4000 μm, and a height of 0.05-1000 μm, optimally 0.05-500 μm relative to said substrate probe region;
and optionally, reactor-protecting structure comprising protective unit with a distance less than 1000 μm, optimally less than 500 μm from said substrate probe-region;
and optionally, 3). marking-system convex, a marker-containing convex that does not cover said probe-ligand,
wherein:
(1). said top plane and bottom plane are parts of top unit and bottom unit of said striped-capillary reaction-chamber, respectively;
(2). said substrate probe-region is on said top plane or/and bottom plane;
(3). said closed partition-structure is placed between said top plane and bottom plane;
(4). material and dimension of said planes, and distance between said top plane and bottom plane are such that the capillary phenomenon of analysis media can take place in said striped-capillary reaction-chamber;
(5). said substrate blank region and substrate probe region are on the same plane of a same substrate, which presents a water-absorptivity less than 0.1 g/g and a contact angle of the surface static water as 40-80°;
(6). said highly-hydrophobic convex contains, at least in its partial surface, highly-hydrophobic material, wherein said highly-hydrophobic material presents a water contact angle of 40° bigger than that of said substrate probe region;
(7). said hydrophobic convex contains, at least in its partial surface, hydrophobic material, wherein said hydrophobic material presents a water contact angle as 55-80°;
(8). said water-absorbing convex contains, at least in its partial surface, water-absorbing material, wherein said water-absorbing material presents a water-absorptivity more than 0.1 g/g;
(9). said highly-hydrophilic convex contains, at least in its partial surface, highly-hydrophilic material, wherein said highly-hydrophilic material presents a water contact angle less than 40°; and (10). said protective unit closes at least partially said reactor structure when no sample is subjected, and it is irreversibly removed completely or partially when sample is to be subjected.

63. The analysis-chip of claim 62,
wherein said convex is formed by solidifying liquid substance or/and fixing solid substance onto surface of said chip,
wherein:
1). said liquid substance includes solution, paint, gel, emulsion which contain said highly-hydrophobic, hydrophobic, highly-hydrophilic and water-absorbing materials respectively; and 2). said solid substance includes plate, film, board, tape, and powder which contain said highly-hydrophobic, hydrophobic, highly-hydrophilic and water-absorbing materials respectively.

64. An analysis-chip, which comprises one or more flow or non-flow reactors with minimal-height,
wherein said reactor comprises at least one or more striped-capillary reaction-chambers with a height of less than 1000 μm, optimally 500 μm, and optionally, reactor-protecting structure, wherein said reaction-chamber comprises:
1). top plane and bottom plane with a width more than 600 μm;
2). probe-ligand and substrate probe-region in which said probe-ligand is immobilized;
3). closed partition-structure with a height of 1-1000 μm, optimally 1-500 μm; and
4). inlet and outlet,
wherein:
(1). said top plane and bottom plane are parts of top unit and bottom unit of said striped-capillary reaction-chamber, respectively;
(2). said substrate probe-region is on said top plane or/and bottom plane;
(3). said closed partition-structure is placed between said top plane and bottom plane;
(4). material and dimension of said planes, and distance between said top plane and bottom plane are such that the capillary phenomenon of analysis media can take place in said striped-capillary reaction-chamber;
and (5). said substrate probe-region presents a water-absorptivity less than 0.1 g/g and a water contact angle as 40-80°.

65. The analysis-chip of claim 62, wherein:
1). said closed partition-structure presents a height of 1-300 μm, optimally 30-100 μm;
2). said top plane presents a width of 1000-15000 μm and a length more than 1000 μm; and 3). said bottom plane presents a width of 1000-15000 μm and a length more than 1000 μm.

66. The analysis-chip of claim 65, wherein said closed partition structure includes one or more of the following reversible or irreversible enclosing structures:
1). thermal enclosing structure;
2). chemical enclosing structure;
3). reversible or irreversible adhesive layer;
4). highly-hydrophobic layer;
and 5). mechanic enclosing unit including coating, plate or tape of elastic polymer.

67. The analysis-chip of claim 64, wherein said top unit or/and bottom unit of said reaction-chamber present a thickness less than 1 mm, optimally less than 0.2 mm, and a detecting-light transparency rate more than 90%.

68. The analysis-chip of claim 64,
wherein said probe-ligand is immobilized on either said top plane or said bottom plane,
wherein said plane with said immobilized probe-ligand presents hydrophilic property whereas said plane without said immobilized probe-ligand presents hydrophobic property.

69. The analysis-chip of claim 64, wherein said top plane is made of glass material whereas said bottom plane is made of hydrophilic or hydrophobic plastic.

70. The analysis-chip of claim 64, wherein one or more said probe-ligands are immobilized in one area whereas one or more ligats of said ligands are immobilized in one other area in said probe region, wherein said ligand and ligate include antigen and antibody.

71. An analysis-chip, which comprises one or more open reactors with minimal-height, wherein said reactor comprises probe-ligand, substrate probe region, open partition-structure with minimal height, and optionally, reactor-protecting structure, wherein said open partition-structure comprises:
1). substrate blank region with a width of 0.5-10 mm;
2). highly-hydrophobic convex or/and water-absorbing convex, all of which presents a height less than 11000 μm, optimally less than 500 μm relative to said substrate probe region, wherein:
(1). said substrate blank region and substrate probe region are on the same plane of a same substrate, which present a water contact angle as 40-80°;
(2). said highly-hydrophobic convex contains, at least in its partial surface, highly-hydrophobic material, wherein said highly-hydrophobic material presents a water contact angle of 40° bigger than that of said substrate probe region;
and (3). said water-absorbing convex contains, at least in its partial surface, water-absorbing material, wherein said water-absorbing material presents a water-absorptivity more than 0.1 g/g.

72. The analysis-chip of claim 62, wherein said highly-hydrophobic convex presents a height of 0.1-100 μm.

73. The analysis-chip of claim 62, wherein said highly-hydrophobic material presents a water contact angle that is over 70° bigger than that of said substrate probe region.

74. The analysis-chip of claim 62, wherein said highly-hydrophobic material presents a water contact angle that is over 90° bigger than that of said substrate probe region.

75. The analysis-chip of claim 62, wherein said highly-hydrophobic material presents a water contact angle that is over 110° bigger than that of said substrate probe region.

76. The analysis-chip of claim 62, wherein said highly-hydrophobic material includes highly-hydrophobic organic material or/and highly-hydrophobic nano-material.

77. The analysis-chip of claim 62, wherein said highly-hydrophobic material includes one or more of the following materials:
1). highly-hydrophobic organosilicon and its derivatives;
2). highly-hydrophobic fluororesin and its derivatives;
3). highly-hydrophobic polymer; and
4). paint or/and solid substance containing highly-hydrophobic nano-particle.

78. The analysis-chip of claim 62, wherein said water-absorbing material includes one or more of the following:
1). natural water-absorbing material;
2). solid porous material of hydrophilic inorganic compound; and
3). water-absorbing material of synthetic polymer.

79. The analysis-chip of claim 78, wherein said water-absorbing material includes one or more of the following: a). capillary-structure-containing paper product, cotton product, or/and sponge as well as their modified materials, b). calcium salt; c). water-absorbing materials based on cellulose or its derivative, d). water-absorbing materials based on starch or its derivative, and e). water-absorbing materials based on synthetic resin as well as compound generated by grafting, blocking and copolymerizing, paper product, cotton product, sponge and its modifier, calcium salt, water-absorbing cellulose material, water-absorbing starch material, and water-absorbing synthetic resin produced by grafting, blocking and copolymerizing.

80. An analysis-chip, which comprises one or more open non-flow reactors with minimal-height,
wherein said reactor comprises at least probe-ligand, substrate probe region, open partition-structure with minimal height, and optionally, reactor-protecting structure, wherein said open partition-structure comprises:
1). substrate blank region with a width of 0.5-10 mm; and optionally, 2). hydrophobic convex with a height less than 1000 μm, optimally less than 500 μm relative to said substrate probe region, wherein:
(1). said substrate blank region and substrate probe region are on the same plane of a same substrate, which presents a water-absorptivity less than 0.1 g/g and a water contact angle as 40-80°; and
(2). said hydrophobic convex contains, at least on its partial surface, hydrophobic material, wherein said hydrophobic material presents a water contact angle of 55-80°.

81. The analysis-chip of claim 62, wherein said hydrophobic convex includes hydrophobic coating.

82. The analysis-chip of claim 81, wherein said hydrophobic coating includes colored hydrophobic line, or/and colored hydrophobic strip.

83. An analysis-chip, which comprises one or more flow reactors with minimal-height, wherein said reactor comprises at least:
1). probe-ligand,
2). substrate probe region with a water-absorptivity less than 0.1 g/g and a water contact angle as 40-80°;
3). convex flow-path comprising high-hydrophilic convex with a width of 5-4000 μm and a height of 0.05-1000 μm, optimally 0.05-500 μm relative to said substrate probe region; and optionally, 4). reactor-protecting structure,
wherein said high-hydrophilic convex contains, at least in its partial surface, highly-hydrophilic material, wherein said highly-hydrophilic material presents a water contact angle less than 40°.

84. The analysis-chip of claim 62, wherein said convex flow-path refers to a coating with said highly-hydrophilic material.

85. The analysis-chip of claim 62, wherein said convex flow-path comprises hydrophobic convex or/and highly-hydrophobic convex as its partition structure, wherein said convex presents a height less than 100 μm, optimally less than 500 μm.

86. The analysis-chip of claim 62, wherein said convex flow-path comprises immobilized separating-media, including electrophoresis media or chromatography media.

87. The analysis-chip of claim 83, wherein said reactor comprises also the open partition structure.

88. An analysis-chip, which comprises one or more reactors with minimal-height, wherein said reactor comprises at least probe-ligand, substrate probe region, marking-system convex, and optionally, reactor-protecting structure, wherein said marking-system convex refers to marker-containing convex that does not cover said probe-ligand.

89. The analysis-chip of claim 62,
wherein said marking-system convex refers to convex of controlled marker-releasing system;
wherein said controlled marker-releasing system comprises marker and presents a half-releasing-period of said marker of more than 10 seconds, optimally more than 30 seconds;
wherein said marker comprises labeling reagent and marking-ligand,
wherein:
1). said labeling reagent includes one or more of the following reagents: enzyme, fluorescent dyestuff, chemiluminescent catalyst, nonferrous metal or nonferrous metallic salt, dyestuff and paint;
and 2). said ligand includes one or more of the following substances: antigen, antibody, biotin, drug ligand polypeptides, DNA, RNA and the fragments thereof.

90. The analysis-chip of claim 89, wherein said controlled marker-releasing system comprises said marker and controlled-releasing agent.

91. The analysis-chip of claim 89, wherein said marking convex presents a height less than 1000 μm, and is fixed around the array of said probe-ligand in said reactor or inside the array of said probe-ligand to form an array of probe-ligand and marking system.

92. The analysis-chip of claim 90,
wherein said convex of controlled marker-releasing system includes mono-sandwiched or multi-sandwiched structure made up of said marker and controlled-releasing agent;
wherein said sandwiched structure refers to structure where the concentration of said marker is higher inside than outside.

93. The analysis-chip of claim 90, wherein said controlled-releasing agent includes water-soluble organic compound or organic compound that will disintegrate in water solution.

94. The analysis-chip of claim 93, wherein said organic compound includes one or more of the following materials: carbohydrate and its derivatives thereof, plant starch and modified starch, plant glue, animal glue, modified cellulose, polymer and condensate.

95. The analysis-chip of claim 62,
wherein said reactor-protecting structure comprises protective unit with a distance less than 1000 μm, optimally less than 500 μm from said substrate probe-region;
wherein said protective unit closes at least partially said reactor structure when no sample is subjected, and it is irreversibly removed completely or partially when sample is to be subjected.

96. An analysis-chip, which comprises one or more reactors with minimal height, wherein said reactor comprises:
1). easily-detachable substrate easily dismantled if needed, or/and
2).reactor-protecting structure with minimal-height, comprising protective unit, wherein:
i). Said height, a distance from substrate probe-region to bottom plane of said protective unit is less than 1000 μm, optimally less than 500 μm;
and ii). said protective unit closes at least partially said reactor structure when no sample is subjected, and it is irreversibly removed completely or partially when sample is to be subjected.

97. The analysis-chip of claim 95, wherein said protecting unit includes one or more of the followings: organic film or/and plate, film or/and plate of metal-organic complex, and slide.

98. The analysis-chip of claim 97, wherein said protecting unit is connected with the reactor through one or more of the following reversible/ irreversible enclosing structures: thermal enclosing structure, chemical enclosing structure and reversible or irreversible adhesive layer.

99. The analysis-chip of claim 97, wherein said protecting unit is precut for the convenience of possible removal.

100. An analysis-chip with a high density of reactors, wherein:
1). said density of reactors is more than 2 reactors/cm², optimally more than 3 reactors/cm² on at least one plane of a substrate; and
2). said reactor comprises partition structure with a height less than 1000 μm, optimally less than 500 μm.

101. The analysis-chip of claim 100, wherein said density is more than 5 reactors/cm².

102. The analysis-chip of claim 100, wherein said reactor refers to said minimal-height reactor.

103. A base-plate, comprising:
1). more than one of substrate probe regions; and
2). one or more of the following reactor structures:
(1). the convex flow-path by claim 62;
or (2). the open partition structure.

104. A qualitative or/and quantitative analysis method, including:
1). subjecting sample into reactor of said analysis-chip and taking probe-selective reaction and marking reaction respectively, wherein said analysis-chip is that of anyone from claim 62;
and 2). washing the reactor and analyzing the result of the reaction.

105. A test device, which comprises cleaning unit,
wherein said cleaning unit is used for cleaning up the residual in analysis-chip reactor when reaction is finished;
wherein said cleaning is performed through absorption with water-absorbing material in said cleaning unit;
wherein said water-absorbing material presents a water-absorptivity larger than 0.5 g/g.
